(19) United States
(12) Patent Application Publication
(10) Pub. No.: US 2020/0306394 A1
(43) Pub. Date: Oct. 1, 2020

(54) METHODS FOR IMPROVING INTEGUMENTARY TISSUE BY TOPICALLY APPLYING ACIDIFYING COMPOSITIONS

(71) Applicant: Avadim Health IP, Inc., Asheville, NC (US)

(72) Inventor: Stephen Thomas Woody, Asheville, NC (US)

(21) Appl. No.: 16/902,144

(22) Filed: Jun. 15, 2020

Related U.S. Application Data
(62) Division of application No. 14/745,091, filed on Jun. 19, 2015.
Application No. 14/745,091 is a continuation-in-part of application No. 14/629,320, filed on Feb. 23, 2015, now Pat. No. 10,046,137.
(60) Provisional application No. 61/943,287, filed on Feb. 21, 2014.
(60) Provisional application No. 62/082,019, filed on Nov. 19, 2014.

(51) Int. Cl. A61L 2/00; A61N 5/10; A61B 10/00; A61K 8/9789; A61K 8/9794; A61K 8/19; A61Q 17/00; A61Q 19/10

(52) U.S. Cl. CPC A61L 2/0035; A61L 2/0088; A61N 5/10; A61B 10/007; A61N 2005/1098; A61K 8/9794; A61K 8/19; A61Q 17/005; A61Q 19/10; A61K 8/9789

(57) ABSTRACT

Method of using an antiseptic and pH modulating solution provides improved health outcomes in part by decolonizing surfaces, animate and inanimate, and improving tissue function and stress response, particularly the barrier properties and antimicrobial properties of tissues, especially skin and muscles, whether damaged or intact and at risk of damage. The method includes initial application followed by continued topical application on a periodic basis until the damaged area is restored or the risk of injury has passed, followed by continued maintenance application for a period of time thereafter. The method of using the solution is believed to restore and improve the functioning of tissues that naturally interrupt pathogenic mechanisms of disease in addition to providing antimicrobial support. The method can be adapted to improve a plurality of health or consumer care outcomes, from preventing or substantially reducing rates of hospital acquired infections to improving muscle performance and recovery, among others.

### CROSS REFERENCE TO RELATED APPLICATIONS

This patent applications a divisional of U.S. patent application Ser. No. 14/745,091, which was filed in the United States Patent & Trademark Office on Jun. 19, 2015, and is entitled Methods for Improving Integumentary Tissue by Topically Applying Acidifying Compositions, which is a continuation-in-part of U.S. patent application Ser. No. 14/629,320, which was filed in the United States Patent & Trademark Office on Feb. 23, 2015, and is entitled Method for Maintenance of Urethral Catheters, which claims the benefit of Provisional Application U.S. Ser. No. 61/943,287, which was filed in the United States Patent and Trademark Office on Feb. 21, 2014, and is entitled Method for Maintenance of Urethral Catheters, Including Steps up to and after Catheter Removal and also claims the benefit of Provisional Application U.S. Ser. No. 62/082,019, which was filed in the United States Patent and Trademark Office on Nov. 19, 2014, and is entitled Method for the Prevention and Treatment of Acne; and incorporates by reference each of these applications in their entirety.

### FIELD OF THE INVENTION

This invention relates to treating surfaces, including mammalian tissues, by topical application of products promoting one or more health benefits, and more specifically to products promoting health in consumer and health care settings.

### BACKGROUND OF THE INVENTION

The variety of sanitizing soaps and cleansers that have been developed to kill viruses, bacteria, and other harmful microbes on skin and on surfaces is legion. In spite of these developments in sanitizing cleansers, the Food and Drug Administration (FDA) says that the most effective way to prevent the transmission of diseases, at least in the consumer antiseptic wash market, is frequent hand washing with soap and warm water, which can be supplemented with a hand sanitizer. A hand sanitizer containing at least 60% alcohol or a persistent antiseptic is recommended if soap and water are not available. Latex or other impermeable gloves provide another layer of defense and frequently are used in health care settings and in food preparation, but are somewhat impractical for use in a variety of other contexts.

There are several examples of the uses of products for sanitizing. The Centers for Disease Control anticipates that on occasion deadly flu viruses may develop, and flu virus easily transmits from one person to another primarily through respiratory droplets dispersed through coughing, sneezing, and touching surfaces or shaking hands. The flu pandemic of 1918, caused by an H1N1 flu strain, was particularly devastating and disproportionately killed younger, healthier persons, potentially due to a cytokine cascade in which an otherwise healthy immune response overwhelms its victims although those with less healthy immune systems are not necessarily impacted to the same degree. For the consumer market, hand sanitizing stations for dispensing no-rinse alcohol-based foams frequently were placed at elevators, escalators, and other locations frequently touched by a wide variety of people during and after the related H1N1 swine flu pandemic of 2009 and in response to the hysteria of the time.

Antiseptic antiviral and antibacterial products are widely used today in both the consumer and health care settings to combat the transmission of viruses and bacteria that can cause disease. Hospitals, physician offices, and extended care facilities frequently use antiseptic antibacterial solutions to clean the skin around wounds and for a variety of other purposes, including preoperative skin preparation or to prepare a patient for a number of other procedures. Two well-known antiseptic antibacterial products also having at least some antiviral activity are chlorhexidine and povidone-iodine solutions, including the well-known BETADINE® brand antiseptic. Alcohol and povodine-iodine solutions frequently are used in the health care setting for pre-injection skin preparation. Swabs of cloth may be impregnated with these solutions and pre-packaged for one-time use in wiping the skin prior to receiving an injection.

Many commonly used cleansing and antiseptic antibacterial compounds are too harsh for wide application and sometimes their use is restricted. Alcohol and chlorhexidine are not generally used on the delicate perineal skin, urinary meatus, and contiguous mucosa and can even contribute to problems on other skin, including the outer layer of the stratum corneum and transitional surfaces between mucosae and stratum corneum generally, including, for example, the lips. Although most all of these cleansing products are capable of reducing potential contaminants, these compounds may actually contribute to subsequent infections by drying the skin, removing naturally occurring lipids, or establishing a basic pH, whereas normal skin has a somewhat acidic pH, moisture content, and lipid structure that serves inter-dependent permeability and barrier functions limiting growth of microorganisms, providing for chemical defense, and controlling moisture loss.

Human skin is at a more basic pH in newborns and as we age. Basic pH is believed to promote the growth of microorganisms. Stripping the skin of its naturally occurring and protective lipids is believed to create micro-abrasions and cause moisture loss through the epidermis, drying the skin. Although initially decontaminating, current antiseptic solutions may initiate a cascade of poor skin conditions enabling viral and bacterial contaminants more readily to enter dry or torn skin and cause infection. Specialty lotions may be applied to restore the skin and are widely used in extended care facilities and neonatal units, but normally are not as effective as healthy, intact, and somewhat acidic skin. There are additional disadvantages for some of the current antiseptic skin solutions. Chlorhexidine solutions cannot be sterilized or otherwise treated by techniques presently approved by the FDA for sterilization. Use of chlorhexidine for surgical site preparation is “off-label” in this regard as the FDA requires products labelled for surgical site preparation to be sterile. Considerable consumer concern has been raised whether chlorhexidine and other related over-the-counter antibacterial and antiseptic hand and body washes are safe and effective as labeled and advertised.

The FDA was sued in 2010 by the nonprofit National Resources Defense Council in the Southern District of New York over the use of triclosan as a topical antimicrobial in over-the-counter (“OTC”) drug products. As a result of the ensuing litigation, the court issued a consent decree in which the FDA agreed to investigate various categories of products in the health care market, but not the consumer market, in which triclosan has been used in a health care setting, including health care personnel hand washes and hand rubs, surgical hand scrubs and hand rubs, and patient preoperative skin preparations.

In its recently proposed rule published May 1, 2015, in the Federal Register at Vol. 80, No. 84, pages 25166 through 25198, the FDA proposes that additional data is needed to support the safety of antiseptic active ingredients generally, not just triclosan, for various categories of products in health care settings. These active ingredients include, but are not limited to, alcohol solutions at 60 to 95%, chlorhexidine, povidone-iodine solutions, triclosan, and others. See page 25171, Table 3—“Eligibility of Antiseptic Active Ingredients for Health Care Antiseptic Uses.” The FDA determined chlorhexidine to be ineligible for inclusion in any of the five health care antiseptic uses. For those included in the list as eligible for use in at least one category, the FDA plans to require additional log reduction data to demonstrate effectiveness in evaluating the benefit-to-risk ratio behind determinations that a drug product containing the active ingredient in question is generally recognized as effective (“GRAE”) (page 25166); additional minimum data under current scientific standards for systemic and long-term exposure to demonstrate safety in evaluating whether the active ingredient is generally recognized as safe (“GRAS”), in part because daily use chronically exposes health care workers to these compounds (page 25167) and in part because the compounds, long in use, can now be tested under current scientific standards for carcinogenic potential, developmental and reproductive toxicity, and other potential effects (page 25181); and the FDA plans potentially to exclude active ingredients from future OTC health care antiseptic final monographs in the event of manufacturer's failure to provide sufficient supporting data for GRAE and GRAS determinations under current scientific standards (page 25167).

The FDA may be responding at least in part not only to chronic exposure to active ingredients of topical antiseptic products among health care workers and some consumers with chronic conditions, but also to the well-documented rise of infections in health care settings, many of which are attributed to overuse of antibiotics and the rise in infections by antibiotic resistant bacteria. Hospitals have reported drastic increases since the early 2000's in infections by antibiotic-resistant Clostridium difficile (C. diff), a common gut bacterium resistant to alcohol-based hand sanitizers that can reproduce unchecked in the gut after antibiotic use has cleared out beneficial bacteria that normally keep C. diff in check. C. diff. is especially problematic in long term care facilities for the elderly in which antibiotic use and incontinence regularly occurs. The predominant strain exhibits a mortality rate three times that of its less virulent predecessors and is being treated in part through fecal transplants to recolonize the gut. C. diff has been estimated to be responsible for 12.1% of health care-associated infections by Shelly S. Magill, M.D. and others in the New England Journal of Medicine in an article published Mar. 27, 2014 entitled “Multistate Point-Prevalence Survey of Health Care-Association Infections.” N Engl J Med 2014; 370: p. 1198.

Other antibiotic-resistant deadly bacterial infections commonly occurring in the hospital setting include methicillin-resistant Staphylococcus aureus (“MRSA”), vancomycin-resistant Enterococcus faecalis (“VRE”), and the even more deadly carbapenem-resistant Enterobacteriaceae (CRE) (Federal Register at Vol. 80, No. 84, p. 25169—“[I]n the health care setting, the potential for spread of infection may be relatively higher than in the U.S. commercial setting.”). CRE is especially deadly and is said to kill up to 50% of infected persons.

One in twenty-five (25) patients in the United States is said now to develop a hospital-acquired infection. N Engl J Med 2014: 370: p. 2001-2002. Approximately ten percent (10%) of those developing an infection died as a direct result at the rate of 205 deaths per day according to the “CDC Technical Information—HAI and Antibiotic Use Prevalence Survey,” available on the World Wide Web at the domain name cdc.gov.

Despite currently prevalent methods and documented efforts by the FDA to update labelling requirements for health care antiseptic products, infections in hospitals and healthcare facilities remain high and impact patient outcomes negatively. OTC consumer antiseptic products, some of which contain active ingredients similar to health care antiseptic products, can be harsh of application. Long-term regular use of harsh products is undesirable and chronic exposure to active ingredients may not be safe or effective.

It would be desirable to develop improved methods of treating the skin using products that could protect the patient or consumer from viruses and bacteria and that also do not present problems associated with chronic exposure or adversely impacting the skin. It would be desirable to develop alternative methods and products to improve generally the support of mammalian tissues, to reduce the incidence of disease transmission, and to improve resistance to infection, by topical application of solutions that do not have or at least reduce the drawbacks associated with the above-described prevalent treatments and products.

### SUMMARY OF THE INVENTION

The invention relates to a method for improving health outcomes, especially tissue function and stress response, including the interrelated and co-dependent permeability barrier and antimicrobial properties of both damaged skin and intact healthy skin at risk for damage along with the underlying skin structures of the integumentary system, the capillary bed, and associated muscle tissue, whether covered by skin or exposed as the result of a burn or other wound. The invention includes new methods for preventing or reducing the incidence, severity, and recurrence of disease by focusing on the etiology of the disease state and interrupting the pathogenic mechanisms that are responsible, including topical applications of substances that support and even improve tissue function. By skin, we mean to include areas of the body covered by the epidermis and its outermost layer, the stratum corneum; the delicate perineum and associated structures; mucous membranes, including the mouth, ear canals, nasal passages or nares, the urethra, and others along with their associated meatuses; and transitional surfaces between mucous membrane and stratum corneum.

The invention is based at least in part on previously unrecognized and unappreciated properties of THERAWORX® brand skin cleanser and related formulations, which is available from the manufacturer thereof and the applicant herein, Avadim Technologies, Inc. in Asheville, N.C. The mechanisms by which the remarkable results reported herein have been obtained by using this skin cleanser in the method of the invention are not yet fully understood although certain results have been documented in support of the invention.

In one embodiment, the method of the invention comprises improving health outcomes by topically applying a formulation as described herein to damaged or intact and at-risk tissues to support and improve tissue function and response, including permeability and antimicrobial barrier properties. The formulation is applied to the damaged tissue or the at-risk tissue initially and on a regular periodic basis for so long as the damage or risk is present and thereafter for a time sufficient to establish a substantially reduced likelihood of recurrence of damage or risk. Providing a continuum of care for a wide array of problems or potential problems is described herein that is believed to enable the remarkable and unexpected results reported. The continuum of care includes the initial application, implementation of a protocol for the problem to be solved, complying with the protocol through regular periodic applications until the damage or risk is resolved, and performing maintenance applications thereafter for a period of time until the probability of recurrence of the damage or risks has passed. The formulation comprises ingredients selected for antimicrobial properties and for acidifying at least the outermost layers of tissue throughout their thickness, improving oxygen uptake, water transport, and elimination of metabolic waste products. Damaged tissue and intact healthy tissue includes muscle and skin at risk for damage, including areas of the body covered by the stratum corneum, mucous membrane, and transitional surfaces between mucous membrane and stratum corneum.

The method of the invention substantially prevents, reduces the likelihood of, and supports the improvement by the skin of compromised barrier and antimicrobial function. In this manner, the method is believed to interrupt the origin or etiology of a disease or prevent the underlying causes of disease that can result in a pathogenic mechanism. One example is the so-called “atopic march,” in which, for example, neonates developing eczema eventually progress in childhood to allergic rhinitis and asthma. Another is the spread and dissemination of deadly antibiotic-resistant bacteria responsible for the relatively recent dramatic rise in hospital acquired infections. The method can be used to treat first, second, and even third degree, full thickness burns in which the epidermis and dermis have been removed, exposing subcutaneous tissues and muscles; to prevent, substantially reduce the risk and intensity of, and treat muscle cramping and soreness following vigorous exercise; and for preventing or reducing the likelihood of viral or bacterial infection on both damaged tissues and healthy, intact tissues, including by extended periodic decolonization steps. The method can be used in connection with skin at risk for infection, as during a hospital stay or in connection with, for example, episodes of incontinence.

The steps of the method comprise topically applying on a regular periodic basis and for so long as the damage or risk is present and thereafter for a time sufficient to establish a substantially reduced likelihood of recurrence of damage or risk a composition as described below comprising ingredients selected for surfactant properties, anti-inflammatory properties, promoting cell growth, enhancing the immune system function, antimicrobial properties, scavenging free radicals, and humectant or emollient properties. Typically, this composition will be antiseptic, having antiviral and antibacterial activity, mildly acidic, and non-antibiotic.

THERAWORX® brand skin cleanser is based upon a formulation described in Harod U.S. Pat. No. 6,358,516 issued Mar. 19, 2002 and entitled One-step System for Cleansing, Conditioning, and Treating the Skin, the contents of which are incorporated herein by reference in their entirety. Previously unrecognized and unappreciated properties of THERAWORX® brand skin cleanser have now enabled the method of the invention to be used in connection with making healthy and intact or damaged tissue more resistant to microbial attack, improving barrier and permeability functions, and mitigating biological mechanisms that can cause disease. THERAWORX® brand skin cleanser is now known to modulate the pH of the tissues to achieve a preferred state of acidic pH over an extended period of time, including lowering the pH over the entire thickness of the outer layer of tissue, increasing oxygen uptake, stimulating healing and transport of waste products, and promoting resistance to viruses and bacteria. THERAWORX® brand skin cleanser is demonstrated to kill envelope viruses including, but not limited to Ebola virus, and can be used in an eight-step protocol, set forth below, including both pre-and post-patient care and health care worker regimens, to prevent or reduce the spread of Ebola, in part by the treatment of clothing and other surfaces with which medical workers may contact infected persons and by pre- and post-procedure decolonization procedures that go beyond simple bathing.

THERAWORX® brand skin cleanser maintains the pH of the stratum corneum, mucosa, and transitional membranes throughout its area of application at an acidic pH from the outermost surface of the skin or membrane to the innermost. At least in the stratum corneum, this means that the THERAWORX® brand skin cleanser solutions as applied alter the “normal” and steeply increasing gradient from the acidic outermost surface to the more alkaline innermost surface adjacent the capillary bed, thus further improving the barrier properties and disease-fighting characteristics of skin regardless of age, pigmentation, or whether stratum corneum, mucosae, or transitional area and whether healthy and intact or not.

Topical application to the skin or to burns and other wounds increases oxygenation of the blood supply, supporting healing; improves heat transfer properties; reduces evaporative cooling through the mechanism of trans-epidermal water loss (“TEWL”); and promotes rapid clearance after exercise of substances accumulating in the underlying muscle tissue that may cause soreness, delaying cytokine response, stabilizing lysosomes, and providing a number of other benefits not limited to its antimicrobial properties or its “balanced pH.” As demonstrated in the detailed description below, the results are remarkable and unexpected. THERAWORX® brand skin cleanser comprises multiple ingredients, described in more detail below, many of which have multiple properties, including anti-inflammatory properties, promoting cell growth, enhancing the immune system function, antimicrobial properties, scavenging free radicals, and humectant or emollient properties. In addition, unlike, for example, chlorhexidine, the ingredients in the formulation can be sterilized by FDA approved techniques, which sterilization or at least treatment to reduce colony forming units below a threshold value, can improve shelf-life and preserve the activity of the other components in THERAWORX® brand skin cleanser solutions, many of them being well-known preservatives in themselves and believed to have antiseptic and antimicrobial properties, including antiviral and antibacterial properties. Radiation generally is believed to be effective and gamma radiation, which is an FDA approved method for sterilization, can be used to produce THERAWORX® brand skin cleanser solutions at a log reduction in colony forming units (CFU's) of from 10⁻⁶ to 10⁻³ or 10⁻² colony forming units (CFU's), as needed or desired. Achieving a sterility assurance level (SAL) of 10⁻⁶ generally meets the most stringent FDA sterilization requirements. The SAL of 10⁻⁶ means a probability of not more than one viable microorganism in an amount of one million sterilized items of the final product.

The pH of the skincare solution used in the practice of the method of the invention is acidic, and should not be so low as to be painful of application nor so high as to promote infection. A suitable range of pH is from about 4.0 to 6.0; a range of 4.4 to 5.5 has been successfully used. Surfactants, to the extent they are used, preferably are zwitterionic so as not to strip naturally occurring and protective lipids from the skin, allowing the skin to stay hydrated. The solution should be antimicrobial, anti-bacterial and antiseptic, and will typically be non-antibiotic, unnecessary antibiotic use contributing to resistance in bacteria strains. It is especially useful if the solution is capable of maintaining the balance of flora associated with normal skin, even as the flora is reduced. It is also desirable that the solution be easily absorbed through the epidermis and at least into the deep layers of the dermis in the absence of creating micro-abrasions that can provide an entry point for infectious agents.

One example of a solution useful in the practice of the method of the invention comprises: lauryl glucoside surfactant; dimethicone as an antifoaming agent also having surfactant action; aloe vera, for its anti-inflammatory properties, to promote cell growth, enhance the immune system response, facilitate absorption, promote healing, and to act as a humectant or emollient; allantoin for similar properties to aloe, including the ability to scavenge free radicals; cocamidolpropyl betaine for its anti-inflammatory properties and action as a humectant or emollient; beta glucan for its properties similar to aloe and allantoin; CITRICIDAL® brand grapefruit extract, having many properties similar to aloe and allantoin; colloidal silver, a well-known absorption facilitator that also facilitates an antimicrobial response and enhances the immune system; Vitamin E for its properties as a humectant or emollient and ability to act as a free radical scavenger; and glycerin for its humectant and emollient properties.

Thus, the invention as described above provides an easily implemented method that approaches the skin barrier and tissues in an entirely new way, acidifying the skin to support and enhance its ability to repair itself from the inside out, recognizing that the skin's many functions are interrelated and co-dependent, including the permeability barrier, the antimicrobial barrier, hydration of the subcutaneous regions, and defenses against ultra violet rays, antioxidants, and mechanical injury. In tissues, even in the case of severe burns, acidification assists in preventing infection and may also increase oxygenation, providing increased opportunity for healing. The variety of uses to which such a method can be put provides for a wide array of applications, including, but not limited to, decolonizing patients in intensive care units; decolonizing patients exhibiting fecal incontinence upon transport from long term care facilities to hospitals to reduce the likelihood of transmission of viruses and bacteria; decontaminating surfaces in rooms in health care facilities, from tables and walls to nonwoven surgical gowns and booties or shoe coverings; treating, improving, and preventing urinary tract infections, catheter acquired and otherwise; application to the T-zone of the face, nose, brow ridge, and seven meatuses to reduce the transmission of viruses and bacteria transferred to the hands or to the face by the hands by touching one's face; and other protocols for health care workers; supporting and improving the skin's own defense mechanisms; treating, alleviating and preventing the recurrence of acne; promoting healing of wounds, including pre-surgical preparation, pre-injection preparation, and third degree full thickness burns; substantially reducing the likelihood of and alleviating soreness in muscle tissue by topical application; delaying cytokine response in the prevention and reduction of sunburn and in response to viral infection; stabilizing lysosomes and reducing trans-epidermal water loss; treating bladder or mouth conditions by accessing the mucus membrane lining and applying the solution as described to substantially reduce the presence of bacteria.

### BRIEF SUMMARY OF THE DRAWINGS

The foregoing and other advantages and features of the invention and the manner in which the same are accomplished are set forth in the following detailed description taken in conjunction with the accompanying drawings, which illustrate preferred and exemplary embodiments and in which:

FIG. 1 is a flow diagram illustrating the steps of the method of the invention generally for improving health outcomes, capable of accomplishing a wide variety of beneficial results in both health care and consumer applications;

FIG. 2 is a modified hub and spoke wheel diagram illustrating the steps of the method of the invention and the various tissues and conditions to which the steps may be adapted to achieve more specific beneficial health outcomes in a variety of consumer and health care settings;

FIG. 3 is a flow diagram illustrating the steps of a method for decolonizing a patient in a health care facility to prevent the spread of disease;

FIG. 4 is a flow diagram illustrating the steps of a method for treating burns to preventing or reducing the risk of infection and to stimulate or at least provide the opportunity for healing;

FIG. 5 is a flow diagram illustrating the steps of a method for improving muscle performance and recovery;

FIG. 6 is a flow diagram illustrating the steps of a method of the invention for preventing or reducing the risk of contamination of a urine specimen;

FIG. 7 is a flow diagram illustrating a protocol for decolonizing the T-zone to facilitate a reduction in disease transmission in health care workers;

FIG. 8 is an illustration of application of the protocol of FIG. 7;

FIG. 9 is a flow diagram illustrating the steps of a method of the invention for preventing or reducing the risk of Ebola infection in health care workers;

FIG. 10 is an illustration of the application of the protocol of FIG. 9 to decolonizing a patient; and

FIG. 11 is a flow diagram illustrating the steps of an eight-step (8 step) method for decolonization.

### DETAILED DESCRIPTION

The invention will now be described more fully hereinafter with reference to the drawings summarized above in which are illustrated some, but not all, of the concepts of the invention. Indeed, the invention may be embodied in many different forms and should not be construed as limited to the specific embodiments set forth herein; rather, the embodiments provided in this disclosure are intended to satisfy applicable legal requirements.

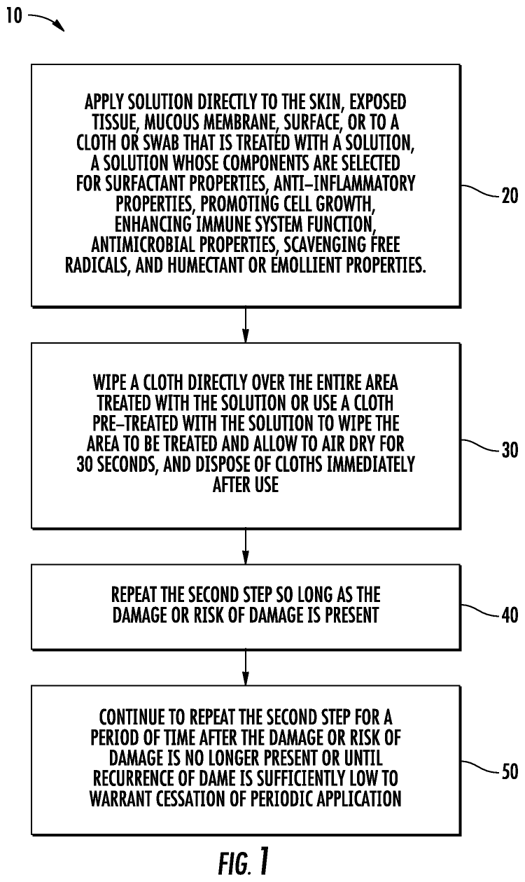

FIG. 1 illustrates generally at 10 the steps of a method of the invention for improving health outcomes in both health care and consumer applications to improve tissue function and stress response. It should be recognized that specific applications may normally have more detailed steps and some of these steps are described below. It is believed that the skilled artisan made aware of the method steps set forth herein will readily consider specific applications based on the particular instance of improved tissue function and response that is sought.

The steps of the method comprise, as illustrated at step 20, topically applying to mammalian tissue or to an inanimate surface a solution of components that are selected and blended for surfactant properties, anti-inflammatory properties, promoting cell growth, enhancing immune system function, antimicrobial properties, scavenging free radicals, and humectant or emollient properties. Tissues can include, but are not limited to, intact or damaged skin as defined elsewhere herein, tissues that the solution impacts through the skin, and exposed muscle tissue, as after an injury or burn. Inanimate surfaces include, for example, medical devices for insertion or placement within living tissue, a nonwoven foot covering or protective gown for a health care worker, or a room surface, generally for the purpose of decolonizing the surface or substantially precluding colonization of the surface with a solution that is not normally irritating to tissues. Depending on the specific application, the solution may be applied as a liquid, a spray, a foam, or from cloths or towels containing the liquid. The solution normally has been treated to substantially reduce the number of colony-forming units (CFU's”) in the solution sufficiently to also substantially reduce the probability of colony-forming units compromising the efficacy of the solution components in the properties described above.

Although some limited benefits can be obtained by a single application of the solution as set forth in step 20, to obtain benefits in accordance with the practice of the invention the solution is applied to the intact or damaged skin, exposed flesh, or inanimate surface on a regular periodic basis as set forth in step 30. In this manner, improvements in tissue function and stress response may be realized, as described below, and viral and bacterial loads reduced below the threshold probability for infection, on both tissues and surfaces.

The mechanism of action of the solution is not entirely understood. It is believed that the mechanism of action includes at least in part modulation of the pH of the skin and tissues at an acidic level of from about 4.5 to 6.0 efficaciously modulating the pH continually if applied about once every 3 to 12 hours, depending on the specific need. For many needs, periodic application every four to six hours is desirable at a pH of from about 4.5 to 5.5.

As set forth in step 40, solution is applied to the skin, exposed flesh, or surface for so long as the damage or risk of damage is present. Specific instances are described below. Thereafter, the solution continues to be applied, step 50, for a period of time after the damage or risk of damage is no longer present or at least until an assessment can be made that the likelihood of initial infection or recurrence of damage is sufficiently low to warrant cessation of periodic application. Normally, the period of application in accordance with step 50 is from a few hours to days to a week or a month.

By skin and exposed tissues, it should be understood that we mean to convey muscle tissue, including tissue exposed as the result of even third degree burns or other injury and all types of skin, including the outermost stratum corneum, the delicate perineum area of the groin, meatuses and associated mucous membranes, and transitional structures including, for example, the lips. The stratum corneum is that outermost layer of skin covering the bulk of the body, comprising the outer layer of the epidermis and the final product of epidermal differentiation. The stratum corneum is essential to formation and maintenance of a cohesive permeability barrier that guards against excessive transcutaneous water loss and serves as an external barrier against microbial attack. Formation of the outer layer of the epidermis is a complex process often referred to as “keratinization.” Keratinization is characterized by, among other matters, water loss and a reduction in pH from the inner to outermost stratum corneum layer, the development of thin overlapping horn cells called corneocytes, specialized cross-linked proteins that are highly chemical resistant, and specialized non-polar lipids that provide a water barrier property as mortar in a bricks-and-mortar like construction with corneocyte bricks.

The population of keratinocytes undergoes continuous renewal throughout life. A mitotic layer of basal cells replaces cells at the surface as they slough off. As they move above the basal layer of the epidermis, keratinocytes undergo the keratinization differentiation process, progressively changing in shape and content and eventually transforming from polygonal living cells to anucleate, nonviable, flattened squames replete with keratin and other proteins. The constant outward movement of corneocytes to be sloughed off at the surface in the process of an orderly desquamation of individual keratinized cells is said to be a built-in mechanism to preclude pathogens from gaining a foothold. Impairment of desquamation, as when the binding force between corneocytes increases under stress and causes the cells to desquamate in clumps, is often characterized as scaling, as in eczema and psoriasis, and treated with everything from skin creams to steroids. Nevertheless, treatments that interfere with the processes of keratinization and desquamation of the stratum corneum inevitably damage the stratum corneum functions, including water permeability and chemical and microbial barrier functions, promoting scaling, redness, pruritus, and decreasing flexibility.

The stratum corneum layer is only about 100 microns thick and even thinner in some other mammals. The stratum corneum displays an acidic pH on its outermost surface of normally 4.5 to 5.0 in healthy skin in human children and adults, somewhat higher in neonates and older adults. Yet in sharp contrast, the innermost layer of the stratum corneum is typically at a pH of about neutral, just under 7.0. Acidic pH is essential for several functions, including inhibiting colonization of the skin and underlying structures, including the more alkaline capillary bed, by pathogenic bacteria, maintaining barrier homeostasis by activating enzymes, and for cohesion of the stratum corneum and an orderly desquamation of cells, preventing premature degradation. Neonates and the elderly typically have skin that displays a more alkaline neutral pH. Compromised buffering capacity of a more alkaline stratum corneum can lead to dermatitis, which can be exacerbated by, for example, urea-soaked skin. Problems in neonates skin, including eczema, the potential for atopic march, and infection are well documented.

One remarkable property we have discovered is that continual application of the THERAWORX® brand skin cleanser solution to the skin periodically and over a period of time alters the pH of the stratum corneum and maintains that pH in a favorable manner. The stratum corneum remains acidified from its outermost layer to its innermost layer at a fairly uniform pH of from about 4.5 to 5.5 or 6.0, depending on the solution and its frequency of application. Maintenance of stratum corneum and other tissue surfaces, including exposed muscle tissue, as in a third degree burn, in an acidic condition over time by multiple periodic applications of the THERAWORX® brand skin cleanser solution provides the opportunity for the skin to improve, to engage its own natural barrier properties, and to ward off bacterial and viral pathogens. Acidification of the entire layer increases oxygenation of the underlying capillary bed, improving its heat transfer properties and enabling faster cooling of muscle tissue and clearance of the waste products of metabolism thought to result in muscle fatigue and soreness after intense exercise. Trans-epidermal water loss (“TEWL”) is reduced, also assisting in reducing the frequency and severity of muscle cramps. Cytokine response is delayed, so the accompanying efforts of the body to respond to disease through swelling, fever, and redness can be reduced and extreme reactions to disease, which can be life threatening, and sunburn, are ameliorated or avoided.

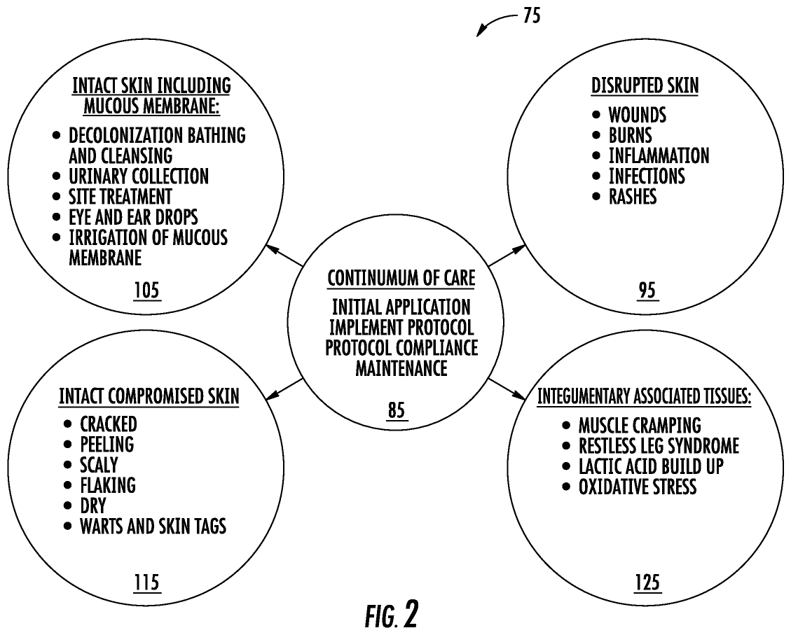

FIG. 2 illustrates generally at 75 in a hub-and-spoke wheel diagram the steps of the method of the invention and the various tissues and conditions to which the steps may be adapted to achieve more specific beneficial health outcomes in a variety of consumer and health care settings. Hub 85 illustrates the core steps of the protocol constituting the method of the invention, following the illustration of FIG. 1, in a continuum of care for the patient or health care worker as the case may be in a health care facility, typically a hospital, extended care facility, or other health care facility. Step 1 is initial application of a solution in accordance with the invention in connection with determining the nature of the particular problem and assessing the damage or risk. For example, in the event a catheter or other mechanical device or foreign body is being inserted into a cavity of the body, and desiring to prevent or reduce the likelihood of a catheter acquired urinary tract infection or other device-related infection, then initial application includes wiping the surface of the catheter or other mechanical device and wiping the area of the skin surrounding the area of insertion prior to insertion and after insertion. Step 2 is implementation of a protocol, which may be, for example, a regimen of continual application on a regular periodic basis sufficient to maintain the surface in an acidic pH so as to reduce the favorability of the environment for bacterial colonization and, in the case of skin and more superficial wounds, to enhance the permeability, chemical and microbial barrier functions.

Step 3 requires compliance with the protocol in that the steps of application are repeated on a continual periodic basis, typically from about every three or four hours to every twelve hours, and for other applications it could be every few minutes based on the application. Specific compliance protocols are set forth below in connection with particular uses to which are put the methods of the invention. In the event of device insertion this typically means that the solution is applied to the external portion of the device and surrounding skin until the device is withdrawn. In the case of third degree burns, for example, nonwoven cloths soaked with the solution are typically applied until the risk of infection has been sufficiently abated. These compliance protocols for application are normally continued until the damage is sufficiently corrected or the risk abated.

Step 4 requires that maintenance of application be continued for a period of time after the damage has been sufficiently corrected or the risk abated to provide a degree of security.

It should be recognized that one or more steps of the protocol 85 may not always be required, as when, for example, the method is used in connection with “clean catch” of a urine sample to avoid contaminating the sample. In a clean catch method, a towel moistened with the solution may be used to clean the groin and the area surrounding the urinary meatus. The hands are also wiped, preferably both before and after cleansing the groin area. At this point, initial application, protocol and protocol compliance have been completed and the urine sample obtained. Under these circumstances, the risk of contamination is abated and reoccurrence of the risk unlikely. Continued maintenance will not typically insure against recurrence and so is considered unnecessary from the standpoint of increasing the likelihood of obtaining a clean urine sample. Nevertheless, from the standpoint of achieving the objective of not transmitting disease, continued maintenance of the hands afterward is advisable.

The continuum of care can be applied to disrupted skin 95, including, but not limited to inflamed skin, infected skin, skin exhibiting a rash or other disruption, and wounds and burns, including superficial wounds and burns and more serious depth injures, including thickness burns that expose muscle tissue for direct application of the solution. Lowering pH by as little as 0.6 is reported to increase oxygenation by 50%, greatly impacting the ability of disrupted skin to heal. The continuum of care can also be applied to intact skin, including the mucous membranes, 105; intact, but compromised, skin 115; and associated integumentary tissues 125. When applied to intact skin 105, the method is primarily used for decolonization techniques to reduce the risk of infection for both health care workers and patients, typically in an eight-step protocol for cleansing the head, including the T-zone and nares, the arms, torso, groin and buttocks, legs, and feet. Health care workers would typically decolonize before and after gowning and may also apply the solution to the exterior of the gown. Urinary clean catch is usually an example of application to intact skin, as is pre-surgical or pre-injection site preparation, the use of ear and eye drops comprising the solution, and irrigation of the mucus membranes, including the urethra and bladder, the nares, and the mouth.

Intact compromised skin 115 is similar to intact skin, but is peeling, cracked, scaly, flaky, or dry and may exhibit warts and skin tags. These types of conditions often require diligent adherence to protocol compliance for repeating application of the solution at selected intervals and for sufficient maintenance to correct the damage, restoring the skin's function and reducing the damage. Application of the solution by the methods of the invention supports recovery by the skin of barrier and permeability properties, which are co-dependent. Integumentary associated tissues 125 include, as defined herein: the epidermis, including the outermost stratum corneum, the underlying dermis, with its capillary bed; adipose tissue, and small muscles; and the muscle beneath the skin, to which the solution properties readily penetrate. For example, topical application of the solution to intact skin and maintenance of an acidic pH over time is thought to increase oxygenation of the muscle tissues, to reduce trans-epidermal water loss, and to speed the clearance of waste byproducts of metabolism, alleviating soreness and fatigue and enabling longer and more intense exercise.

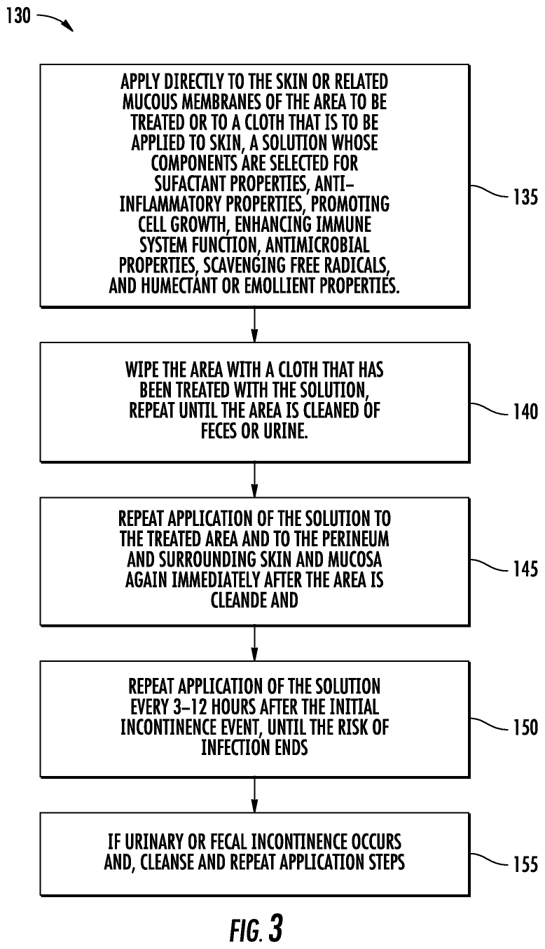

FIG. 3 is a flow diagram illustrating generally at 130 the steps of a protocol of the method directed to decolonizing a patient or healthcare worker in a health care facility to prevent the spread of disease. While we have referred to patients and healthcare workers, the invention could be practiced by concerned individuals or for family members who are experiencing a disease outbreak at their home, such as influenza. Decolonization may be performed on selected areas of the body or on the entire body as when interrupting the spread of particularly serious diseases including flus having a high mortality rate, Ebola virus, and MRSA, VRE, and CRE bacteria having a high mortality rate. In step 135, a solution as described or otherwise meeting the requirements of the invention, is applied to the skin and mucus membranes, if needed. Typically, this is an eight step process that involves using separate solution-impregnated cloths to clean various parts of the body. Swabs may be used to apply the solution to the nares, in particular, to reduce the opportunity for infection by inhaling virus or bacteria, and also the ear canal and the mouth. For some diseases, very few organisms ae required to colonize a full-blown deadly infection. For example, in the case of most strains of H1N1 flu virus, 10² to 10⁹ virus particles are required to cause infection, while for Ebola, hardly any is required by comparison to influenza.

In the event of an episode of incontinence of a patient, then decolonization may only involve the affected area. Typically, solution is applied and the area cleaned after each episode of incontinence, step 140. On transferring a patient from an extended nursing care facility to a hospital, it can be beneficial to decolonize the entire body of the patient and the health care worker. Immediately after the initial cleansing, it is advisable to clean the area a second time, step 145. If the risk or actuality of infection remains, as in the case of an indwelling mechanical device, then the application of the solution should be repeated every three or four to twelve hours until the risk of infection or actual infection has abated, step 150. In the event of a second or any subsequent episode of incontinence, cleansing is repeated, step 155. Normally, an additional step of continuing to apply the solution will be employed for a maintenance period thereafter in those situations where the risk of recurrence or infection remains afterward as when, for example, an indwelling catheter is removed.

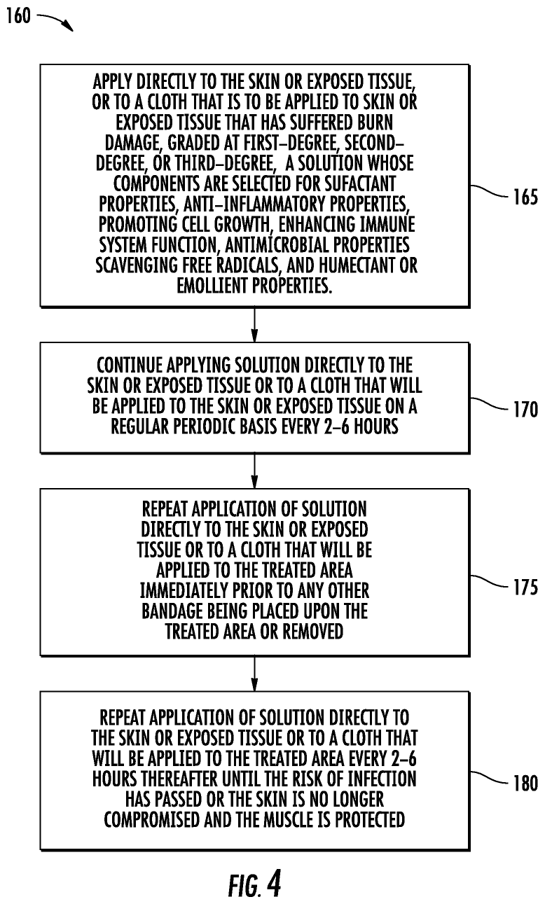

FIG. 4 illustrates generally at 160 the steps of a method for preventing or reducing the risk of infection in a burn and for stimulating healing. The solution is applied in accordance with the invention at step 165 to a first, second, or third degree thickness burn. First degree burns are similar to wounds and may irrigated with a liquid solution or cleansed with an impregnated cloth. Reducing the pH of the burned tissues, oxygenation of the tissues, and antimicrobial activity reduces the likelihood of infection and increases the ability of the tissue to repair itself. Continuing application of the solution to the injury, step 170, on a periodic basis every two to six hours increases the probability that no infection will occur and that the tissue will repair. If a bandage or wound dressing is to be applied, then it is usually beneficial to apply the solution immediately prior to bandaging the wound, step 175. If muscle is exposed, as is the case in third degree thickness burns, then the solution is typically applied by soaking a cloth or bandage and laying the cloth or bandage on the area, and repeating this step every two to six hours, step 180. A health care worker or someone having skill in the art recognizes that changing burn dressings is highly specific and depends on the severity of the burn. For example, to treat a sunburn, the dressing maybe changed more frequently every few hours, for a third degree burn the dressing may be left on the injured area for a matter of days depending on the discretion of the physician or health care worker.

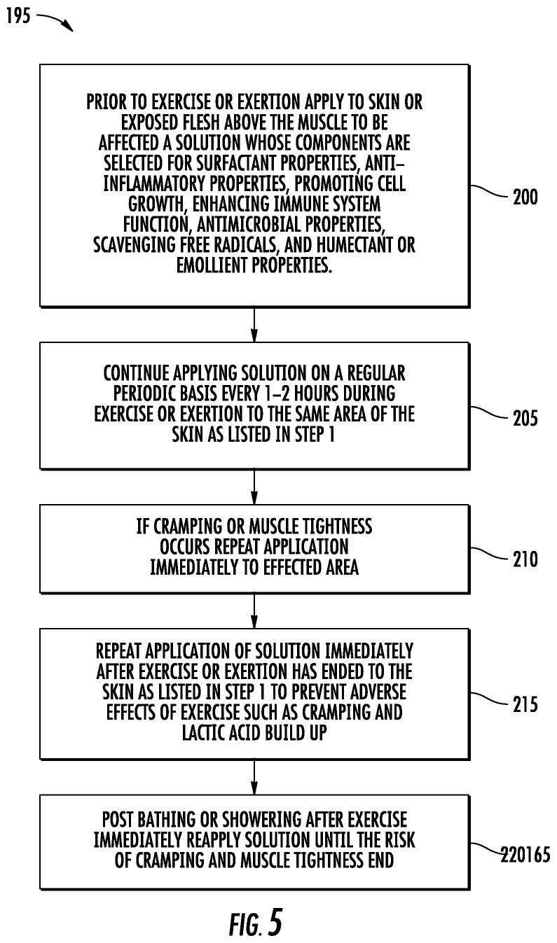

FIG. 5 is a flow diagram illustrating generally at 195 the steps of a method for improving muscle performance and recovery. Active persons, sports enthusiasts and elite athletes alike may apply the solution in accordance with the invention to the skin prior to exertion, step 200. Although the solution can be initially applied after exertion, more pronounced benefits can be realized if the oxygenation and metabolic processing capacity of the muscle tissue is well supported and enhanced prior to exertion. The solution can be reapplied every 1 to 2 hours during exertion, step 205. For some types of athletic events, for example boxing, it might be desirable to apply THERAWORX® brand skin cleanser solution or similar solutions every few minutes between rounds and for other types of athletic events the application could be lengthened to every thirty minutes to an hour or more depending on the nature of the activity, for example in an 18 hole golf game it might be desirable to apply the solution after the 9^{th} hole and again after the 18^{th} hole. If cramping or muscle tightness occurs, then the solution is normally immediately applied at that time, step 210. After exertion is concluded, then the solution is applied yet again, step 215, and again after showering or bathing, step 220, or until the risk of cramping is abated.

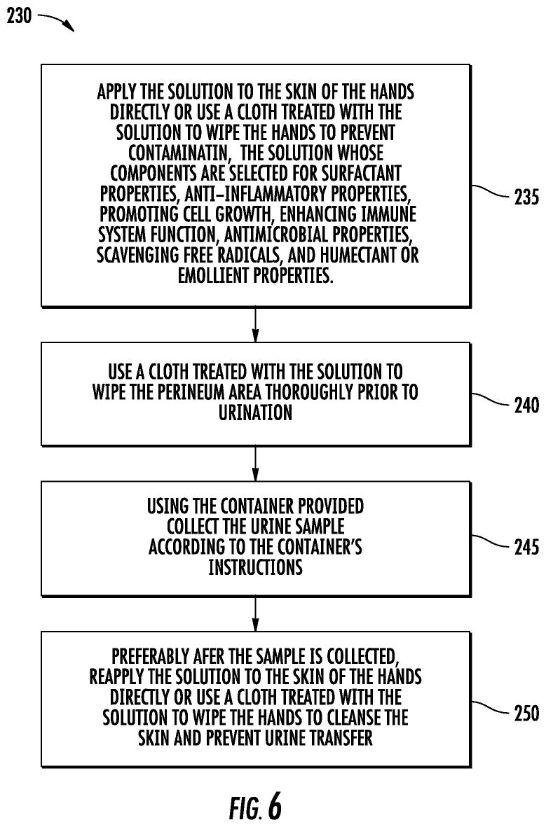

FIG. 6 is a flow diagram illustrating generally at 230 the steps of a method of the invention for preventing or reducing the risk of contamination of a urine specimen. In accordance with step 235 the first step is to apply the solution to the hands, usually with an impregnated cloth wipe. Second, the perineal area is thoroughly wiped with a fresh cloth, step 240, basically to decolonize the delicate perineum, including the urinary meatus and contiguous areas. Typically, it is not necessary to cleanse the hands again prior to collecting the urine specimen, although if repeating step 235 is undertaken, then a fresh cloth is used. Thereafter, the sample is collected, step 245. Once the sample is collected and sealed, then no further cleansing is required to provide a clean catch urine specimen. However, reapplying the application to the hands for hygienic reasons is certainly preferred, step 250.

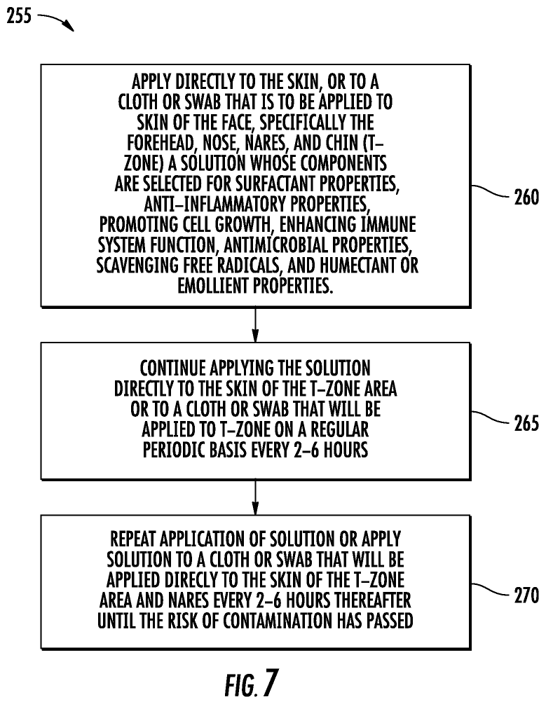

FIG. 7 is a flow diagram illustrating generally at 255 a protocol for decolonizing the T-zone to facilitate a reduction in disease transmission in health care workers, patients, or concerned individuals. The T-zone is so-called after the “T” formed by the horizontal brow ridge to the ears and the vertical line from the nose to chin. The face has seven (7) entrances to the body within the T-zone, comprising the two ear canals, the nares or two nostrils, the two eyes, and the mouth. Each of these provides an especially vulnerable meatus to a mucous membrane and surrounding tissue though which virus and pathogenic bacteria readily may enter, especially through the eyes, mouth and nares. Application of the method of the invention in accordance with the T-zone requires applying the solution directly to the skin of the face, step 260, especially including the T-zone, and is usually the first area to be decolonized in a decontamination effort. Impregnated cloths are especially useful. Thereafter, the solution is applied from swabs generally to the ear canals, nares and around the mouth. A dilute solution of THERAWORX® brand skin cleanser may applied to the inside of the mouth, generally as a liquid to be swished around, spat out, and disposed of in a biologically responsible matter as bio-hazardous material, as is true for any impregnated cloth used for application in accordance with the invention under any of the protocols, with the possible exception of exercise-induced applications in which no risk of infection is present.

In accordance with step 265, application is continued directly to the T-zone about every 2 to 6 hours for as long as the risk of infection is present, step 270, as, for example, in a high risk environment for transmission, especially by respiratory droplets in the case of H1N1 flu, or blood products in the case of Ebola.

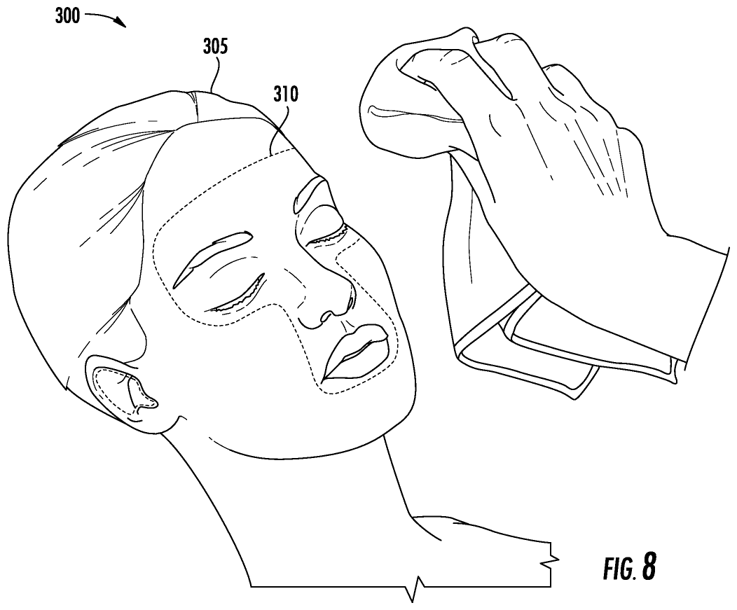

FIG. 8 is an illustration generally at 300 of the application of the protocol steps of FIG. 7 to the face 305 and in particular the T-zone illustrated in dashed outline at 310 and including the right and left ears, right and left eyes and brow ridge, nose and chin, and nares, or nostrils. Generally, the T-zone is wiped with a cloth, although for the ear canals and nares, typically a swab is provided. Disposable one-time use swabs and impregnated towels would be particularly advantageous.

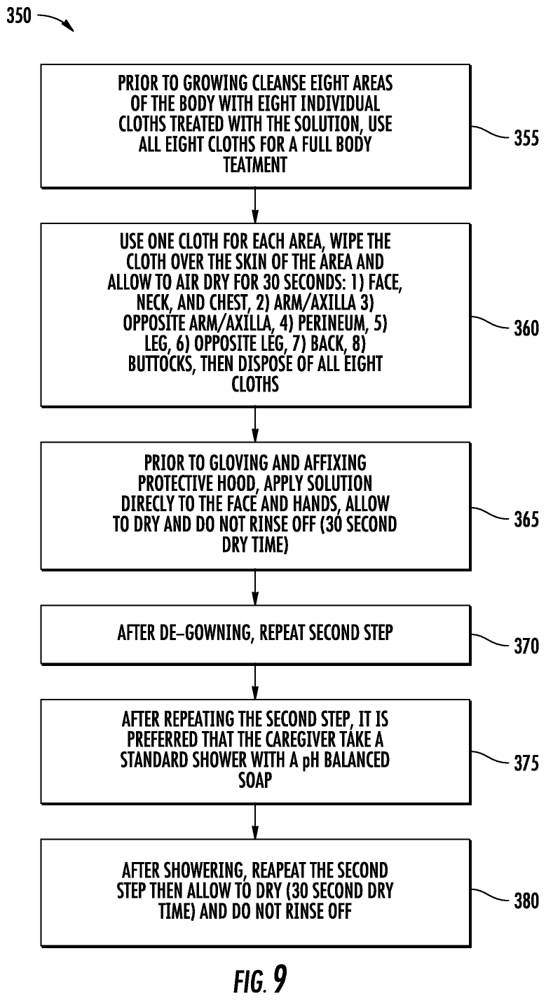

FIG. 9 is a flow diagram illustrating generally at 350 the steps of a method of the invention for decolonizing an Ebola health care worker. The methods of decolonizing a patient and a healthcare worker differ in that decolonizing a health care worker includes 355 pre- and post-gowning procedures. Initially, of course, the health care worker bathes with soap and water and dries with a clean towel. Thereafter, a protocol 360 is implemented for decolonizing the bathed health care worker, typically by using a pack of disposable towels for cleaning each of the major body parts: the head, including the T-zone; the arms, independently; the torso; the legs; the buttocks; and groin. Then, prior to gloving and affixing a protective hood the solution is applied directly to the face and hands 365. Thereafter, the health care worker is gowned using the donning procedures established by the CDC or other pertinent body. The gowns and other coverings may be sprayed or dipped into a solution, as when a fully donned Ebola health care worker, having had THERAWORX® brand skin cleanser or similar solution applied to the exterior clothing, steps fully donned from a dressing area or tent and dips his or her feet into a tub of solution to coat the nonwoven protective booties. Ebola and many viruses are thought to be spread in part by the action of gravity in the treatment area, in which the floor of the treatment area becomes highly contaminated as blood and other body fluids fall from the patient. Similarly, on returning, the Ebola health care worker is carefully disrobed, using the CDC-established doffing procedures, and can repeat cleaning again each major part of the body 370, bathing with soap and water 375 and thereafter repeating the personal care decolonization of the eight major areas of the body 380.

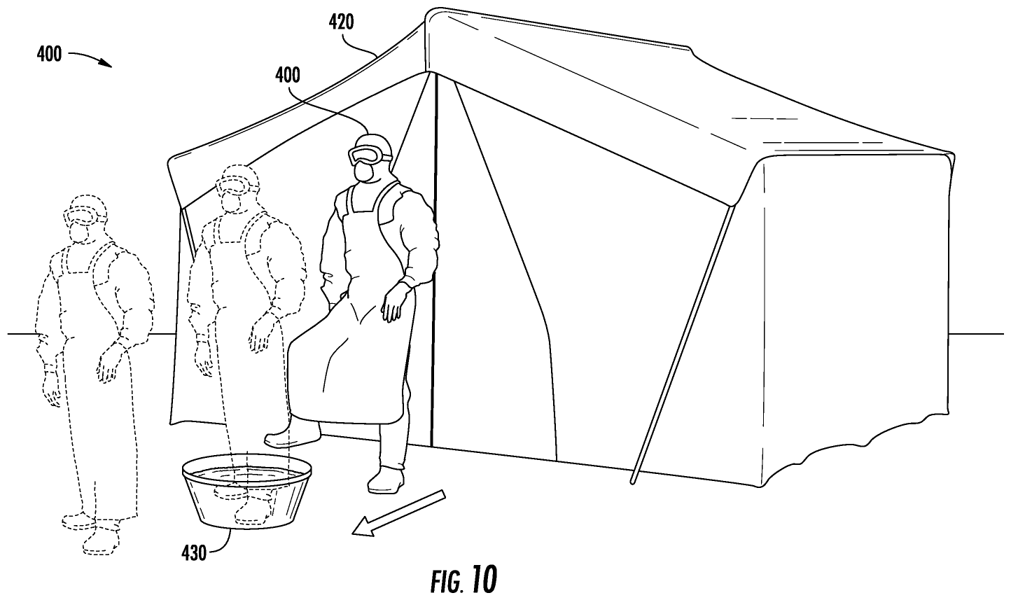

FIG. 10 illustrates generally at 400 the protocol of FIG. 9 applied to a fully donned healthcare worker 410 exiting a robing tent 420 in the field and stepping into a wash basin 430 containing THERAWORX® brand skin cleanser solution or a related solution;

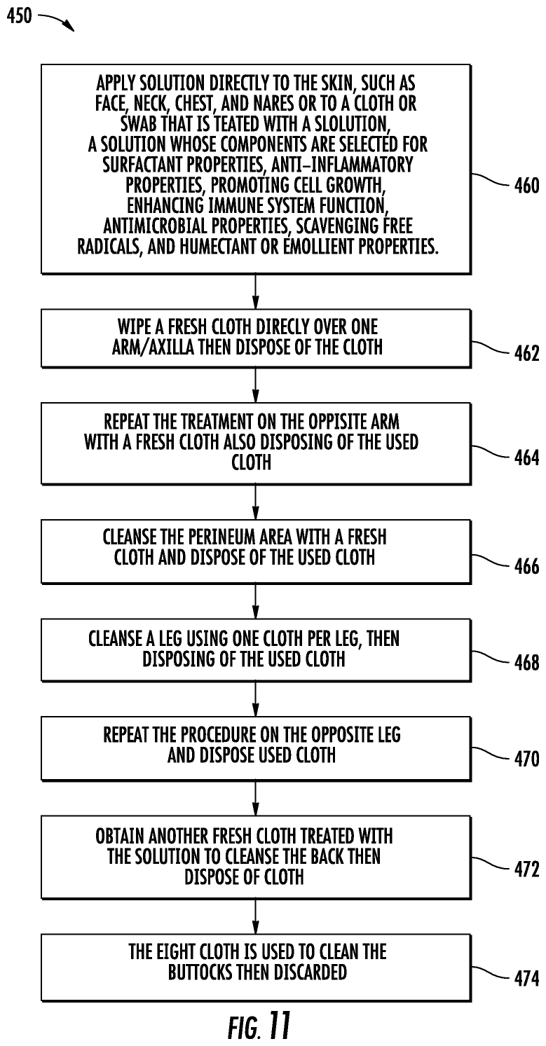

FIG. 11 illustrates generally at 450 a flow chart for the protocol for decolonization of high risk patients and health care workers for Ebola and other highly contagious diseases having a high mortality rate, including, but not limited to, antibiotic resistant bacteria MRSA, VRE, and CRE. Generally speaking, it is useful to provide a convenient pack of eight (8) disposable pre-moistened nonwoven cloths and disposable swabs for the nares. In the first step, step 460, a pre-moistened cloth is used to clean the face, neck, and chest and disposed of, then disposable swabs are used for the nares. A fresh cloth is applied to one arm and the underarm or axilla, step 462, and disposed of, and then the opposite arm and axilla is cleansed with a third fresh pre-moistened cloth, step 464. After the arms, the perineum is cleansed with a fourth fresh cloth, step 466. Thereafter, the legs are cleaned, first one and then the other, each with a fresh cloth, steps 468 and 470. It is helpful to clean the legs in the same order in which the arms were cleaned and to establish the protocol to begin with the same arm each time for consistency and to avoid missteps. For example, if the protocol starts always with the right arm at step 462, then step 468 should be cleaning the right leg. Thereafter another fresh cloth is obtained in connection with step 472 and the back cleansed. The eighth cloth is used to cleans the buttocks last, step 474.

It is helpful and increases absorption and effectiveness of the solution to warm it before application, most especially, although not exclusively, in connection with high risk decolonization. Not only is the solution more pleasant for the patient or health care worker to whom it is applied, but the increase in absorption improves penetration and effectiveness thereby. Typically, the solution or the pre-moistened wipes are heated to an average of about 105° F. in warmer boxes especially adapted to carry the pouches of pre-moistened cloths. The solution should not be heated over about 125° F. for safe, comfortable, application to the skin. The used cloths may be disposed of in the pouch from which they were taken, if desired.

Our previous and co-pending Patent Application Woody U.S. Ser. No. 14/629,320, Method for Maintenance of Urethral Catheters, filed Feb. 23, 2015, recognizes that catheter acquired urinary tract infections can be prevented or substantially reduced in frequency, occurrence, and reoccurrence, by initially decolonizing the delicate perineum, the urinary meatus, and the contiguous mucosa surrounding the catheter insertion site and maintaining these delicate areas in a state that resists infection. The method includes the steps of pre-treatment of the perineum, urinary meatus, mucosa, and the exterior portions of the catheter with THERAWORX® brand skin cleanser and related solutions as described therein both before and immediately after catheter insertion, followed by continual maintenance treatments with the solution at regular intervals of from about 4 to 12 hours for maintenance of the perineum and exposed portions of the catheter. In addition, treatment is performed after each incidence of incontinence. In some embodiments, treatment is also performed immediately before catheter withdrawal and the perineum, urinary meatus, and contiguous mucosa wiped with soft, lint free cloth impregnated with the solution immediately after withdrawal. Still further embodiments may include post-withdrawal maintenance treatment steps in which the perineum, urinary meatus, and mucosa are continually wiped at regular intervals for a sufficient period of time after withdrawal to reduce or preclude infection attributable to the use of a catheter. Typically, this maintenance step is performed by patient self-care after discharge from a hospital for about five to seven days. It should be understood that patients include pets and other mammals that may be catheterized and that patient self-care is performed by a caregiver for animals and humans not having capacity to do so, including newborns and some elderly.

Somewhat similarly, our previous and co-pending provisional application U.S. Ser. No. 62/082,019, entitled Method for the Prevention and Treatment of Acne, filed Nov. 19, 2014, sets forth a protocol for treating various forms of acne comprising, in a twenty-four (24) hour period the method steps of 1) cleansing the skin with a suitable substance as described below, rinsing the skin with water, and drying the skin; 2) applying the substance to the skin and allowing the substance to dry in air and remain on the skin; 3) repeating step (2) at selected intervals of applying the substance to the skin and allowing it to remain in contact with the skin; and 4) repeating step (1). For example, in a specific embodiment, step (1) is accomplished in the morning on arising; step (2) is accomplished immediately after step (1); step (3) is repeated at selected intervals of from 3 to 6 hours throughout the day following step (2); and step (4) is accomplished in the evening on retiring. Of course, depending on one's schedule, steps (1), (2), and (3) may be performed in the evening and step (4) the following morning.

Steps (1) and (4) alone may be sufficient in some instances. In alternative embodiments, the steps may be repeated for as long as desired to prevent acne or reduce its likelihood and severity when it does occur, or the steps may be applied directly to acne lesions for as long as needed to improve the condition of the skin.

It has now been recognized as illustrated above that similar concepts can also be applied to a number of different areas where active infections or damage otherwise may occur to tissues more generally or where tissues are at risk for infection or damage, beyond preventing or reducing the incidence and severity of CAUTI's and acne. The invention has application to wounds more generally, including burns; other types of catheters, including intravenous catheters and catheters used in veterinary medicine; decontamination of environmental surfaces; and decolonization regimens to prevent or reduce health care acquired infections, whether hospital acquired or acquired in an extended-care facility. For example, the invention is useful for preventing or substantially reducing the risk of urinary tract infections generally, whether catheter acquired or not. The skilled artisan apprised of this invention and provided examples of protocols for particular disease or risk states should recognize that initiation of application, regular periodic application for the duration of the damage or risk, and, in many instances, maintenance for a period of time after the damage or risk has been resolved, are useful steps in the practice of the invention.

One solution useful in the practice of the invention is described in Harod U.S. Pat. No. 6,358,516 issued Mar. 19, 2002 and entitled One-step System for Cleansing, Conditioning, and Treating the Skin, the contents of which have been incorporated herein by reference in their entirety. Although the Harod patent recognizes that the solution described therein should be “pH balanced” and have an acidic pH similar to that of skin, and that the solution has antimicrobial properties and is skin compatible, the Harod patent does not recognize or suggest the remarkable results demonstrated by the data examples contained herein. Indeed, it is not yet possible to elucidate the precise mechanism of action.

The solution according to the Harod patent comprises at several ingredients, including eight agents, all eight of which agents are required to be different from each other and are present in relatively small amounts: a surfactant and a humectant or emollient, an anti-inflammatory agent, and antifoaming agent, an agent for promoting cell growth, an agent for enhancing the immune system, an antimicrobial agent, agent to facilitate absorption, and an agent to scavenge free radicals. Many of the compounds listed in the Harod patent have one or more or even several of these functions, although it is believed that the plurality of ingredients exhibits a synergistic impact that is particularly useful in the methods of the invention. Generally speaking the solution exhibits the following properties believed to be relevant to the applications claimed herein: antiseptic properties and properties of supporting the barrier function of the stratum corneum, which includes the ability to reduce and maintain pH at a level of from 4.0 to 5.5 or 6.0 over an extended period of time, especially when used in the protocols of the invention to provide regular continual periodic applications. Generally speaking, the solutions are non-antibiotic but antimicrobial, mildly acidic to pH about 4.0, and zwitterionic when used with a surfactant ingredient for cleansing that is non-polar and does not strip tissues of beneficial lipids. These solutions promote antimicrobial properties in the absence of damage to the skin and muscle tissue; promote healing of existing wounds and burns; and create a zone of inhibition around the wound or burn or decolonized region to preclude recontamination or infection or at least substantially reduce the likelihood of contamination and infection.

The zone of inhibition is a complex phenomenon recognized in connection with the invention that includes not only lowering the pH of the tissue to inhibit bacterial colonization, but orderly desquamation of epithelial tissues in a well-maintained bricks-and-mortar structure of keratinized cells, proteins, enzymes, and a lipid matrix. Unlike normal skin, which has a steep pH gradient from the acidic outer surface of the stratum corneum to the considerably more alkaline pH of the inner surface of the stratum granulosum, skin to which the solution is applied on a continual regular periodic basis has been determined to exhibit a relatively uniform acidic pH throughout its surface. This uniform pH is thought to “super-normalize” the skin, enhancing blood flow in the capillary system of the largest organ in the body, increasing oxygenation of the underlying muscle tissue and the skin, promoting clearance of metabolic products, promoting moisture barrier functions to avoid trans-epidermal water loss, and altering the pH of the skin adjacent normally neutral-to-alkaline blood, which blood tends to promote bacterial growth otherwise if exposed to the elements unprotected by fully functioning skin. Damaged stratum corneum is said to be capable of losing up to 6 liters of water per day by trans-epidermal water loss and may allow ingress of chemical agents and pathogens. Thus, the fully acidified stratum corneum enhances the ability of the skin to ward off infection.

It should be recognized that by “antiseptic” is meant a substance that kills and prevents growth and reproduction of bacteria, protozoa, yeast, fungi, and viruses. The term “antiseptic” is sometimes used synonymously with “antimicrobial,” which is how we used the term here. Antiseptics and antimicrobials should be distinguished from antibiotics, which also kill bacteria. The solution used herein typically is antiseptic.

Potential solutions that are antiseptic and antimicrobial, having a pH of from about 4.0 to 6.0, and including the functionalities of being anti-inflammatory, antifoaming, cell growth promoting, immune system enhancing, antimicrobial, absorptive into the skin, and scavenging free radicals include mixtures of aloe, dimethicone, allantoin, cocamidolpropyl betaine, citrus-based extracts including Citricidal® brand quaternary compound derived from grapefruit, colloidal silver, and vitamin E; mixtures of aloe, dimethicone, allantoin, and colloidal silver or grapefruit extract; mixtures of dimethicone, allantoin, grapefruit extract, colloidal silver, and vitamin E; mixtures of aloe, dimethicone, cocamidolpropyl betaine, grapefruit extract, and colloidal silver; mixtures of aloe, dimethicone, grapefruit extract, and colloidal silver; and mixtures of dimethicone, cocamidolpropyl betaine, colloidal silver, and Beta-glucan.

The cocamidolpropyl betaines and dimethicone have surfactant properties, as does lauryl glucoside. At least dimethicone also has antifoaming properties. Cocamidolpropyl betaines also have anti-inflammatory properties. Aloe has anti-inflammatory, cell growth promoting, immune system enhancing, absorption facilitating, healing promoting, and humectant and emollient properties. Allantoin has anti-inflammatory, cell growth promoting, immune system enhancing, free radical scavenging, and healing promoting properties. Beta-glucan has cell growth promoting, immune system enhancing, absorption facilitating, free radical scavenging, and healing promoting properties. Grapefruit extracts have immune system enhancing, antimicrobial, and free radical scavenging properties. In addition, polyphenolics, bioflavonoids, pyncogenol, and grapeseed extract may be used for some of these functions in the formulation or to supplement the other ingredients.

Some formulations may include additional ingredients, possibly performing preservative functions inhibiting microbial and fungal growth and extending shelf life: methyl- and propyl-parabens. Additional related compositions that may potentially be useful in the practice of the invention include those disclosed in Huckfeldt et al. U.S. patent application Ser. No. 13/095,708, filed Apr. 27, 2011, and entitled Composition for Skin Sanitization and Protection and Method for its Use, and Bevilacqua et al. U.S. patent application Ser. No. 14/385,752, filed Sep. 16, 2014, and entitled Compositions and Uses of Antimicrobial Materials with Tissue-Compatible Properties, the contents of which two applications are incorporated herein by reference in their entirety.

Thus, a skincare and cleansing agent useful in the practice of the invention may include, as described in U.S. Pat. No. 6,358,516, among other ingredients:
(a) at least one surfactant;
(b) at least one anti-inflammatory;
(c) at least one anti-foaming agent;
(d) at least one cell growth-promoting agent;
(e) at least one fast-acting antimicrobial agent, each of said ingredients being skin-compatible and different from the other ingredients of said composition; and at least one different ingredient selected from the group of:
(f) immune system-enhancing agents, wherein at least one immune system-enhancing agent is aloe vera, beta glucan, colloidal silver, or allantoin;
(g) absorption facilitation agents, where in at least one absorption facilitating agent is beta glucan, aloe vera, or colloidal silver;
(h) humectants and emollients, wherein at least one humectant or emollient is aloe vera, vitamin E, or cocamidopropyl;
(i) free radical-scavenging agents, wherein at least one free radical-scavenging agent is a bioflavonoid, a polyphenolic compound, a grapefruit-derived quaternary compound, beta glucan, allantoin, vitamin E, pycnogenol, or grape seed extract; and
(j) healing promoting agents, wherein said ingredients are selected to form a stable, no-rinse, radiation-sterilizable composition that air-dries quickly when applied to the skin and that cleanses, therapeutically conditions, and treats the skin in a one-step application, wherein at least one healing-promoting agent is aloe vera, allantoin, or beta glucan.

It should be recognized that where a compound is mentioned in two different categories that the compound serves both functions in the formulation and that each function is present when the compound is present.

A key component of the invention is the ability of the solution to be treated under methods use for sterilization, including, for example radiation. The ingredients used within the formula all are compatible with radiation, including gamma and e-beam radiation. However, the ingredients may also be compatible with other sterilization techniques approved by the FDA including, but not limited to, dry heat, ethylene oxide gas, steam, hydrogen peroxide gas plasma, and ozone. The ingredients may also be compatible with novel treatments not currently considered by the FDA including, chlorine dioxide, ethylene oxide-in-a-bag, high intensity light, microwave radiation, sound waves, ultraviolet light, and vaporized chemical sterilizing systems. According to the Centers for Disease Control, “Any item, device, or solution is considered to be sterile when it is completely free of all living microorganisms and viruses.” The definition is categorical and absolute, meaning an item is either sterile or it is not. A sterilization procedure is one that kills all microorganisms, including high numbers of bacterial endospores. Nevertheless, from an operational standpoint, a sterilization cannot be so categorically defined. Rather, the procedure is defined as a process, after which the probability of a microorganism surviving on an item subjected to treatment is less than one in one million (10⁻⁶). This is referred to as the “sterility assurance level.” A description of various sterilization techniques mentioned is detailed below.

In practice of the current invention, sterilization treatment methods preserve the efficacy of the ingredients of the solution and allow ingredients with antimicrobial properties to expend their energy fighting organism outside of their container, instead of inside the container acting themselves as a preservative and losing efficacy.

Essentially, having antibacterial products in a gamma treated solution allows those antibacterial products to maintain their efficacy because they are not attacking organisms within the solution itself. This in effect extends the shelf life of the ingredients. If the solution was not sterilized the bio-burden would increase and the efficacy of the solution in terms of fighting antimicrobial activity would decline.

In the practice of the invention, gamma radiation of the Theraworx brand solution at 35 kGy has been determined to provide an efficacious solution of assured sterility at 10⁻⁶ to 10⁻⁸, but the intense radiation yellows the product. Preferred is 10⁻² or 10⁻³ to 10⁻⁶. A radiation dose of 4 to 7 kGy typically reaches 10⁻².

In the practice of the invention, gamma radiation of the THERAWORX® brand skin cleanser solution at 35 kGy has been determined to provide an efficacious solution of assured sterility at 10⁻⁶ to 10⁻⁸, but the intense radiation yellows the product. Preferred is 10² or 10⁻³ to 10⁻⁶. A radiation dose of 4 to 7 kGy typically reaches 10⁻².

The methods in which the solution can be used have expanded, and specific examples based on Theraworx brand cleansing solutions in support of the protocols set forth in the drawings are provided below. Generally speaking, the composition improves the normal functions of skin and thereby improves permeability and antimicrobial barrier properties, which are interrelated and co-dependent, for both damaged skin and intact healthy skin at risk for damage. By interrelated and co-dependent, we mean that both the permeability barrier function and antimicrobial and chemical barriers are improved and that both are necessary to healthy functioning of the tissues. A compromised permeability barrier not only contributes to excessive trans-epidermal water loss, but also provides ingress for bacteria, viruses, and chemical attack. A compromise antimicrobial barrier can result in an infection, which compromises the permeability barrier.

The methods in which the solution can be used have expanded, and specific examples based on THERAWORX® brand skin cleanser solutions in support of the protocols set forth in the drawings are provided below. Generally speaking, the composition improves the normal functions of skin and thereby improves permeability and antimicrobial barrier properties, which are interrelated and co-dependent, for both damaged skin and intact healthy skin at risk for damage. By interrelated and co-dependent, we mean that both the permeability barrier function and antimicrobial and chemical barriers are improved and that both are necessary to healthy functioning of the tissues. A compromised permeability barrier not only contributes to excessive trans-epidermal water loss, but also provides ingress for bacteria, viruses, and chemical attack. A compromise antimicrobial barrier can result in an infection, which compromises the permeability barrier.

Application of the composition in accordance with the invention can substantially prevent, reduce the likelihood of, or support the reversal by the skin of compromised permeability barrier and antimicrobial function by applying the composition to damaged or at-risk tissues initially and upon a regular periodic basis for so long as the damage or risk is present, and continuing the application until such a time that the risk of damage is deemed sufficiently passed. The method of continual regular periodic application allows penetration into the deeper layers of the tissue, including the capillary bed and the underlying muscle tissue. This method of application, applying the composition initially and on a periodic basis and for a time after until the risk for damage has subsided, can impact the interactions of the skin and muscle to prevent and substantially reduce the severity of muscle cramps, the “lactic acid” threshold, trans-epidermal water loss, and muscle recovery, to name a few.

The stratum corneum plays a key role in many physiological pathways. By improving the functioning of the stratum corneum, even that of intact skin, we allow the antimicrobial and co-dependent permeability barriers to function at an enhanced level. The antimicrobial activity of the skin and the barrier repair permeability are inseparable and their enhancement or restoration influence many factors including stratum corneum hydration, ultra-violet defense, antioxidant defense, mechanical defense, and the neurosensory interface. By recognizing these mechanisms, previously not recognized in the use of related solutions for skin compatible cleansing to avoid the harsh impact of soaps, other antiseptics, and the like, we have created a pathogenesis-based therapy enabling benefits not previously realized that include improvement and enhancement of stratum corneum characteristics, the properties of mucous membranes, meatuses, transitional areas between the stratum corneum and mucous membranes, and muscle tissue. The methods of the invention mitigate biological mechanisms that can lead to a diseased state in healthy skin or assist in repairing skin that is already damaged.

The basic concepts behind the steps of the methods of the invention are suitable for multiple applications including direct application as a liquid, foam, gel, or impregnated cloth to intact skin, disrupted skin, mucous membranes, transitional areas, meatuses and muscle tissue.

Applications to intact skin not only enhance the skin's function but also decolonize the skin of harmful bacteria and viruses without negatively impacting the balance of beneficial flora. Suggested applications to intact skin where decolonization would be useful include: catheter care, bathing intensive care patients in procedures specifically designed for decolonization and maintenance in a decolonized state; urinary collection for reducing the likelihood of sample contamination; decolonizing the perineum and surrounding areas, particularly after an incidence of incontinence or prior to inserting a urinary catheter; T-zone decolonization, including the seven openings to the body that provide unique and frequently used pathways for viral and bacterial infection, especially the nares; pre-operative and general application to the nares when aerosolized infectious agents are anticipated; pre-operative site preparation; pre-injection site preparation; prophylactic decolonization for patient transfer, as from an extended care facility to a hospital, upon admission to emergency care, or upon transfer from emergency care to intensive care; site preparation and maintenance of a central line patch; neonatal and elderly adult decolonization and skin enhancement where skin pH is known to be on the alkaline side and at a level that could promote microbial colonization and infection; decolonization following any episode of fecal and urinary incontinence to prevent disease; hand decolonization, decolonization of patients during end-of-life care; decolonization of infected or potentially infected tissues post-mortem; decolonizing foot care and especially diabetic skin care for improving the function of the thicker stratum corneum characterizing the feet and the likelihood of infection associated with higher glucose near-surface capillary blood supply in the feet, which is known to promote infections, including cellulitis and the like; initial, continual periodic, and maintenance cleansing to avoid the chronic itching associated with pruritus; feminine wipes and daily care; baby wipes and daily care; body deodorants for chronic odor control; eye drops for mammals; conjunctivitis; ear drops for mammals; oral care for mammals; initial, continual periodic, and maintenance application to warts and skin tags; shampoos; makeup removers; shaving creams; application to the skin in the event of episodic pseudofolliculitis barbae; initial, continual periodic, and maintenance applications in the use of facial cleansers, cosmetics, primers and the like to avoid or treat and reduce the recurrence of acne and the like.

The invention may also be applied to parts of the integumentary system that are disrupted or exposed, including, for example, muscle and capillary tissue in wounds and burns. The method of the invention may be applied to the initial, continual periodic, and maintenance application treatment of atopic and contact dermatitis, impetigo, acne, diabetic ulcers, venous stasis ulcers, pressure ulcers, mouth ulcers, dermatosis, excema, cellulitis, treatment of a C-section incision site, episiotomy incision site, diaper rash, hemorrhoids, rosacea, skin that has been compromised by laser or radiation treatments or burns, including first, second, and third degree burns, blister care, wound debridement, poison ivy rash, shingles lesions, chicken pox lesions, hives, insect bites, toe nail fungus, and inflammation.

One unique aspect of the invention is its efficacy on mucous membranes, a sensitive type of tissue that is especially susceptible to harsh ingredients. The invention has potential uses for irrigation of the bladder, colon, vagina, nares and nasal passages, and rinsing of the oral cavity. The method of the invention as applied to burns where the skin and its integrated and associated tissues have been damaged also see benefits from enhancing the skin's normal functions. Applications to burns includes first-degree, second-degree, and third-degree burns, as well as sunburns on the skin. The method of the invention of initial, continual periodic, and maintenance application to the tissues enhances barrier repair therapy by reducing the pH to prevent infection and increase oxygen uptake. These two functions of reducing the pH and increasing oxygen uptake speeding healing, keeping skin and muscle tissue healthy and able to fight off a potential infection until such time as a skin graft can be provided or the skin otherwise repaired.

The invention may also be applied to the integumentary system via intact skin to influence the associated tissues, including muscles and the capillary system. Application to the skin can impact interactions between muscle tissue and the layers of the skin. Appreciable effects may be achieved for relieving muscle cramping, trans-epidermal water loss, reducing lactic acid, reducing inter-muscular inflammation, reducing exercise-induced heat, increasing range of motion, speeding transport of excretion products of muscle metabolism, oxidative stress capacity, restless leg syndrome, and neuropathy.

In somewhat more detail, Theraworx brand and similar solutions are believed to affect muscle tissue in the following three ways, all related to initial, continual regular periodic, and maintenance lowering of the skin's pH and enhancing anti-inflammatory response. First is the limiting of trans-epidermal water (“TEWL”) loss. Second is improved oxygenation, and third is improved transport of waste products and reduced inflammatory response. Lowering the pH of the skin limit TEWL. The average person loses 1.5 to 2.0 liters of water a day through the skin. Perspiration makes the pH of the skin go up. TEWL increases as more water is lost through the skin and increases the risk of cramping. Using Theraworx to help regulate the pH of the skin allows limiting TEWL and can relieve cramping.

In somewhat more detail, THERAWORX® brand skin cleanser and similar solutions are believed to affect muscle tissue in the following three ways, all related to initial, continual regular periodic, and maintenance lowering of the skin's pH and enhancing anti-inflammatory response. First is the limiting of trans-epidermal water (“TEWL”) loss. Second is improved oxygenation, and third is improved transport of waste products and reduced inflammatory response. Lowering the pH of the skin limit TEWL. The average person loses 1.5 to 2.0 liters of water a day through the skin. Perspiration makes the pH of the skin go up. TEWL increases as more water is lost through the skin and increases the risk of cramping. Using THERAWORX® brand skin cleanser to help regulate the pH of the skin allows limiting TEWL and can relieve cramping.

There is more blood in the skin than any other part of the body. THERAWORX® brand skin cleanser and related solutions lower the pH of the stratum corneum, thereby impacting the capillary bed to carry and enabling more transport of oxygen and waste products, which assists in reducing inflammatory response. Colloidal silver, when used as a component of the formulation, is believed to reduce the inflammatory response in traumatized muscle and to inhibit cytokine production, thus limiting the impact of cytokine cascade reactions, including an inflammatory cascade. The particle size of the silver in THERAWORX® brand skin cleanser is small enough for trans-dermal migration into muscle tissue. Silver inhibits cytokine production. Cytokines contribute to the inflammatory cascade.

### EXAMPLES

Thus, THERAWORX® brand skin cleanser and related solutions have many properties similar to non-steroidal, anti-inflammatory sparing products, or “NSAID,” but without the negative impacts. NSAID's, including aspirin, ibuprofen, and naproxen, are among the most anti-inflammatories in the world, frequently used for a variety of pain relief, but present multiple risks, including gastrointestinal problems, high blood pressure, and kidney damage.

### Example 1: Muscle Tissue

The following examples document the use of THERAWORX® brand skin cleanser and related solutions as described in various specific protocols of the method of the invention.

The University of South Carolina tested Theraworx brand solution in connection with athletic performance and in a protocol of the invention under outcomes-based standards for use in treating cramps, recurrence of cramps, and muscle tightness. To date, the University report 50 instances of muscle cramping were reviewed with a current success rate of 82% for relieving cramps in under two minutes, and in 73% of those the relief came under one minute of application. Of the reported events of cramping and muscle tightness, only one athlete reported that the incidence of cramping returned and there were no reported returns of muscle tightness after application. Future studies are proposed are planned to test the potential benefits of application in regards to lactic acid removal, muscle tightness, muscle fatigue/muscle failure rate, elimination or soreness, total body effect, overall endurance, muscle/skin temperature and decolonization efficacy and are described below. Muscle tightness limits maximal output and performance. To study muscle tightness, an athletic trainer will selected athlete based on self-reported “tightness” in the hamstring or calf region and apply a solution under the protocol of the invention to the affected hamstring or calf prior to their pre-game or pre-workout stretch routine. The athlete's experience will be evaluated under the MacNab criteria, both before the application of the solution and after the application of the solution and exercise routine.

The University of South Carolina tested THERAWORX® brand skin cleanser solution in connection with athletic performance and in a protocol of the invention under outcomes-based standards for use in treating cramps, recurrence of cramps, and muscle tightness. To date, the University report 50 instances of muscle cramping were reviewed with a current success rate of 82% for relieving cramps in under two minutes, and in 73% of those the relief came under one minute of application. Of the reported events of cramping and muscle tightness, only one athlete reported that the incidence of cramping returned and there were no reported returns of muscle tightness after application. Future studies are proposed are planned to test the potential benefits of application in regards to lactic acid removal, muscle tightness, muscle fatigue/muscle failure rate, elimination or soreness, total body effect, overall endurance, muscle/skin temperature and decolonization efficacy and are described below. Muscle tightness limits maximal output and performance. To study muscle tightness, an athletic trainer will selected athlete based on self-reported “tightness” in the hamstring or calf region and apply a solution under the protocol of the invention to the affected hamstring or calf prior to their pre-game or pre-workout stretch routine. The athlete's experience will be evaluated under the MacNab criteria, both before the application of the solution and after the application of the solution and exercise routine.

Again a study is proposed where the Theraworx brand solution is used in connection with athletic performance and in a protocol of the invention, an athletic trainer will select athletes for testing the Theraworx brand formulation in connection with resistance exercise under the protocol of the invention. Resistance exercises will be selected to impact the same muscles on each side of the body so that one side could serve as the control. The athlete's experience will be evaluated under the MacNab criteria, both before the application of the solution and after the application of the solution and resistance training to failure. Initially, an athlete will perform a set of resistance bicep curls, hamstring curls, and calf curls to failure and the perception of a burning sensation in the muscle recorded. Theraworx brand solution will be applied in accordance with the invention to one side of the body only, to the calf, bicep, or hamstring as the case may be, and the exercise repeated to failure. Thereafter, the athletes will report on the difference between the perceived burning sensations in each body part impacted by the curl exercise.

Again a study is proposed where the THERAWORX® brand skin cleanser solution is used in connection with athletic performance and in a protocol of the invention, an athletic trainer will select athletes for testing the THERAWORX® brand skin cleanser formulation in connection with resistance exercise under the protocol of the invention. Resistance exercises will be selected to impact the same muscles on each side of the body so that one side could serve as the control. The athlete's experience will be evaluated under the MacNab criteria, both before the application of the solution and after the application of the solution and resistance training to failure. Initially, an athlete will perform a set of resistance bicep curls, hamstring curls, and calf curls to failure and the perception of a burning sensation in the muscle recorded. THERAWORX® brand skin cleanser solution will be applied in accordance with the invention to one side of the body only, to the calf, bicep, or hamstring as the case may be, and the exercise repeated to failure. Thereafter, the athletes will report on the difference between the perceived burning sensations in each body part impacted by the curl exercise.

In yet a third proposed study the use of Theraworx brand solution will be evaluated under the MacNab criteria in connection with athletic performance and in a protocol of the invention for treating inflammation. Improving oxygen flow, enhanced thermoregulatory function, and increased transport of waste metabolic product minimizes inflammation. The athletic trainers will select athletes to evaluate Theraworx brand solutions' impact on inflammation. The protocol requires the athlete to apply the Theraworx product before, during, and after the workout as well as the following morning. The athlete will then report the perceived impact based on the MacNab criteria.

In a fourth proposed study, Theraworx brand solution will be studied under the MacNab criteria for evaluating an athlete's perception of maximum energy output. Research indicates that a brief cooling period between sets of exercise results in an increase in the maximum energy output. Exercise induced intramuscular heat is a significant factor in limiting muscle performance. An athletic trainer will select an athlete performing strenuous exercise and in-between sets apply Theraworx brand solution over the entire area impacted by the exercise. The athlete then performs another set and provided feedback regarding his or her perception of energy output. It is believed that the application of the methods of the invention to exercise will result in significant muscle cooling.

### Example 2: Antimicrobial Efficacy

A commercial laboratory tested Theraworx brand solutions for antimicrobial effectiveness using a procedure to determine a five-year real-time aged sample. Five challenge microorganisms were used, including Escherichia coli, Pseudomonas aeruginosa, Staphylococcus aureus, Candida albicans, and Aspergillus brasiliensis. 8 mL of sample Theraworx brand solution were aseptically transferred to sterile tubes for each challenge microorganism. The 8 mL portions were inoculated with 0.1 mL of the respective challenge microorganism and were mixed thoroughly, so that the final concentrations of the test organisms per mL were 1.0×10⁵ to 1.0×10⁶ colony forming units (CFU). The inoculated samples were stored in sterile test tubes to prevent desiccation and were incubated at 20 to 25° C. Plate counts were performed for each inoculation formulation at Days 7 and 14 with a 14 day re-challenge incorporated into the test. Plate counts were repeated at Day7, 14, and 28 of the re-challenge. At each assay interval 0.1 mL of the sample was directly plated. 0.1 mL of each sample were transferred to a sterile tube along with 9.9 mL of sterile Lactobacilli agar (AOAC). The individual tubes were vortexed thoroughly for 30 seconds and serial dilutions of the extract were plated (via pour plate methodology) with tempered/molten tryptone soya agar (TSA) or sabouraud dextrose agar (SDA) containing neutralizers (0.1 Tween 80 & 0.05% Lecithin). The plates were incubated at 30 to 35° C. for 72 hours for bacteria and 20 to 25° C. for 5 to 7 days for fungus.

The results of the test indicated a log reduction of 4.28-5.00 for the bacterial and fungal test microorganisms by Day 7 of the initial test for the 5-year real-time aged samples. There was no increase in the level of microorganisms seen for the remainder of the 28 day re-challenge test, with the exception of Pseudomonas aeruginosa. At the 28 day re-challenge test all organisms exhibited a 99.99% reduction except Pseudomonas aeruginosa, which exhibited a 99.98% reduction, still a very significant reduction.

ASTM E640-06 Standard Test Method for Preservatives in Water-Containing Cosmetics with a 56 day re-challenge was used to test the effectiveness of preservatives in Theraworx in the following organisms: Methicillin resistant Staphylococcus aureus, Escherichia coli, Candid albicans, Aspergillus niger, Pseudomonas aeruginosa, and Staphylococcus aureus. The substrate used was Vitro-Skin® an advanced testing substrate that effectively mimics the surface properties of human skin. It has been formulated to have topography, pH, critical surface tension, and ionic strength similar to human skin. The results of the testing showed that initial inoculations and re-inoculations ranged from >10⁷ organisms to >10⁹ organisms. The preservative in Theraworx reduced bacterial counts by 10⁵ in all organisms and maintained this level of protection throughout the 56 day test regimen even with bacterial re-exposure. Exact kill rates may be even higher as culture plates exhibited no growth after exposure to the solution.

The antibacterial properties of Theraworx brand solutions have been tested against Vancomycin resistant enterococcus faecalis (VRE). Theraworx demonstrated a >99.99% (>4.80 log₁₀) reduction of VRE following a 15 minute exposure time when tested at an ambient temperature of 20.9° C.

Theraworx has was tested for antibacterial effectiveness against Klebsiella pneumonia carbapenem resistant bacterium following a 15 minute exposure and a 99.2% reduction (2.08 log₁₀) following a one hour exposure time, when in the presence of a 5% bovine serum organic soil load and tested at ambient temperature of 20.7° C. Under identical conditions Theraworx was also tested against Escherichia coli carbapenem resistant bacterium and demonstrated a >99.9% reduction (3.84 log₁₀) following a 15 minute exposure and a 99.99% reduction (4.01 log₁₀) following a 1 hour exposure time.

Theraworx was also tested for its duration of action in terms of its antimicrobial performance against Methicillin resistant Staphylococcus aureus (MRSA) using a collagen based inoculation model. Bovine collagen was prepared and divided into three groups: control (normal saline), alcohol based skin cleanser, and Theraworx brand solution. The collagen was placed in the assigned solution and allowed to saturate for five minutes. All specimens were then removed and allowed to air dry for five minutes on sterile paper with each specimen being turned over to facilitate even air drying at the 2.5 minute mark. After drying they were placed in a sterile lidded specimen container. At designated intervals of 15 minutes, 30 minutes, 60 minutes, 120 minutes, and 180 minutes, ten samples from each group were subjected to inoculation using 10⁶ MRSA followed by incubation for 24 hours. Punch biopsies were then performed from the center of each specimen and quantitative cultures performed. The results indicated that at all time intervals in both the control and alcohol based skin cleanser that the MRSA were too numerous to count and appeared to have spread to cover nearly the entire specimen container. However, the results also showed that Theraworx brand solution was effective >99.99% at all time intervals.

In junction with the Texas Biomedical Research Institute (“TBRI”), a biosafety level 4 laboratory registered with the Department of Health and Human Services CDC Select Agent Program, Theraworx brand solution was tested against Ebola, an envelope virus. In the first experiment Vitro-Skin® test substrate was inoculated with a metered dose of the Zaire ebolavirus (EBOV) and the samples were incubated for 5 minutes. The substrate was then wiped with a cloth saturated with Theraworx brand solution and allowed to incubate for five additional minutes. Thereafter, the substrate was cultured with a growth medium-saturated swab to detect infectivity in the host cells. The results showed no infectivity after wiping. It is assumed that the mechanical action of wiping combined with the known anti-viral activity of Theraworx brand solution was sufficient to remove or inactivate the virus.

A second experiment was conducted to evaluate the effectiveness of Theraworx brand solution alone, without wiping, against EBOV. Again EBOV was applied to the test substrate, followed by a 5 minute incubation. The substrate's surface was sprayed with Theraworx brand solution until saturated. After an additional five minute incubation period, the surface was cultured and evaluated for infectivity. The results were present as viral plaque-forming units per milliliter (PFU/ml), indicating level of infectivity. When compared to untreated samples, Theraworx spray treated samples showed a reduction of infectivity of 99.85%.

### Example 3: In Vitro and In Vivo Compatiability

Theraworx has also been tested for in vitro and in vivo biocompatibility using the ISO Intracutaneous Reactivity Test, the ISO Acute Systemic Injection Test, the ISO Guinea Pig Maximization Sensitization Test, and for cytotoxicity the MEM-Elution using L-929 Mouse Fibroblast Cells (ISO). These tests demonstrated the safe use of Theraworx brand solution in contact with breached or otherwise compromised skin. Theraworx is considered non-toxic and non-irritating to the skin and tissues and not to elicit a sensitization response. Additionally, no potential toxic effects as a result of a single-does systemic injection were observed.

Based on the above results, multiple hospitals, health care facilities and sports organizations are performing internal studies to determine if Theraworx brand solutions should be used at their locations for decolonization, prevention of bacterial contamination of urine cultures, efficacy of Theraworx brand solutions in bath wipes for the reduction of skin colonization with VRE in children undergoing hematopoietic stem cell transplantation, perineum decolonization in high-infection rate pre-term premature rupture of membranes (“PPROM”) among pregnant women at risk for this condition, and urine and fecal urinary tract infections not due to a catheter.

### Example 4: Military Field Applications

United States military forces in South Korea tested the product as a field treatment. Using the MacNab criteria, the results were evaluated and rated by the troops as: (1)—poor, no perceived effect; (2)—fair, some perceived positive effect; (3)—good, noticeable perceived positive effect; and (4)—excellent, significant perceived positive effect. Twenty-six (26) respondents evaluated the product in four areas: sanitation; scrapes, cuts, and burns; fungus, jock itch, athlete's foot; and muscle discomfort. More than 80% of respondents rated the field treatment as good with noticeable perceived positive effects in relation to sanitation and to scrapes, cuts, and burns.

The Macnab criteria is a well-established and documented tool used in clinical research and discovery when evaluating the effectiveness on pain of prescription drugs and medical devices. The Macnab criteria provide a results-based assessment of the patient's response to treatment, and in particular, the patient's experience of efficacy or not, apart from the mechanism of action of the drug or device.

Seventy-nine percent of respondents also rated the field treatment as good, with noticeable perceived positive effects, in relation to fungus, jock itch, and athlete's foot. A majority of respondents also rated the field treatment as good, with a noticeable positive perceived effect on muscle discomfort, 63%. When the respondents were asked whether they would use the product again, 92.3% replied “yes.” Additional results are listed in the table below:

TABLE 1

| | 2+ | 4 | 3 | 3+ |
|---|---|---|---|---|
| Sanitation | 46% | 42% | 88% | 96% |
| Scrapes, Cuts, Burns | 52% | 30% | 83% | 100% |
| Fungus (Jock Itch/ | 53% | 26% | 79% | 95% |
| Athletes Foot) | | | | |
| Muscle Discomfort | 42% | 21% | 63% | 84% |

### CAUTI'S

Example 1: A trial was conducted at First Health Moore County Regional Hospital, a 395 bed facility, in Pinehurst, N.C. from August to October 2013. Prior to the trial, First Health's CAUTI rate was similar to other similar size hospitals in North Carolina. After reviewing the CAUTI's that occurred at First Health, it was discovered that the majority of CAUTI's occurred after a Foley catheter had been in place for greater than 5 days, which lead the hospital to believe that it had issues with the care and maintenance of catheters. The hospital then used Theraworx® brand solutions in July of 2013 in its intensive care unit, or ICU, and began the trial in August. Hospital personnel were directed to apply Theraworx® brand solution to the perineum as a pretreatment step, both before and immediately after insertion as described hereinabove and to implement a maintenance treatment step every 8 hours while the catheter was inserted.

In the same time period a year earlier the hospital had 4 CAUTI's in the ICU for 1728 catheter days for a rate of 2.3 infections per 1000 catheter days. During the trial in which Theraworx® brand solution was used in accordance with the invention, the hospital had 0 CAUTI's in the ICU over 1667 catheter days, which is a rate of 0 infections per 1000 catheter days.

The results of this study were reviewed by hospital ICU staff and the product was approved for hospital wide implementation. The policies for catheter insertion and catheter care were reviewed, best evidence based practice was reviewed, and the hospital's policies and protocols were revised in December 2013 for staff to perform catheter care prior to and immediately after insertion and every 8 hours thereafter for the period of indwelling using Theraworx® brand foam cleanser on cloths applied to the perineum and catheter, including the additional step of wiping the perineum with Theraworx® brand cleanser after removal of the catheter.

Example 2: A trial, using the Theraworx solution, was conducted at five intensive care units at Baptist Hospital, a 383 bed facility in Lexington, Ky. The purpose of the hospital study was to determine whether the use of a colloidal silver impregnated wipe and foam cleanser, which was the Theraworx® brand solution, when used as part of a cleansing protocol within the current Foley catheter care protocol practiced by the hospital would be efficacious in reducing the incidence of CAUTI's in the intensive care setting.

Mean infection rates in the five ICU's in 2012 ranged from 1.2 infections per 1000 device days to 5.9 infections per 1000 device days. The hospital performed the steps of the protocol starting in April 2013, including cleansing the perineum prior to insertion with Theraworx® and allowing the solution to dry in air for 30 seconds, opening the sterile Foley catheter and cleansing the Foley catheter with Theraworx®, wiping the meatus with Betadine®, and inserting he catheter using the accepted aseptic techniques. The meatus, perineum, and exposed portions of the catheter were again cleansed with Theraworx® after insertion. Theraworx® soaked cloths were used two to three times daily for maintenance wiping and additional wiping was done as a final cleansing for incontinence. As a result, zero CAUTI's were reported in four out of five ICU's by the second month of the study and by the fourth month all five ICU's had reduced their CAUTI infections to zero infections per 1,000 device. Although some units had achieved zero CAUTI infection rates prior to the start of the study, it was only after the study was initiated that all five intensive care units maintained a zero CAUTI infection rate for the same month. These results exceeded the 2012 mean CAUTI rates and were below the National Healthcare Safety Network CAUTI benchmark of 1.4 infections per 1,000 device days.

Table 1, below, summarizes the results of the hospital study from June 2013 through July 2013 and includes the rates of CAUTI's of each of the five ICU's from January 2013 through July 2013 and the 2012 mean rate of CAUTI's in each of the five ICU's. The corresponding results are illustrated graphically in FIG. 3.

TABLE 1

| | 2012 | Jan- | Feb- | | | | | |
|---|---|---|---|---|---|---|---|---|
| | Mean | uary | uary | March | April | May | June | July |
| 2ICU CAUTI | 2.3 | 5.6 | 0 | 11.2 | 6.8 | 6.6 | 0 | 0 |
| Rate (per 1,000 | | | | | | | | |
| device days) | | | | | | | | |
| 3IN CAUTI | 1.2 | 0 | 0 | 0 | 0 | 0 | 0 | 0 |
| Rate (per 1,000 | | | | | | | | |
| device days) | | | | | | | | |
| 3IS CAUTI | 4.7 | 0 | 9.8 | 0 | 0 | 0 | 0 | 0 |
| Rate (per 1,000 | | | | | | | | |
| device days) | | | | | | | | |
| 4IN CAUTI | 5.9 | 13.5 | 0 | 0 | 0 | 0 | 0 | 0 |
| Rate (per 1,000 | | | | | | | | |
| device days) | | | | | | | | |
| 4IS CAUTI | 0.9 | 0 | 0 | 0 | 0 | 0 | 0 | 0 |
| Rate (per 1,000 | | | | | | | | |
| device days) | | | | | | | | |
| NHSN CAUTI | 1.3 | 1.4 | 1.4 | 1.4 | 1.4 | 1.4 | 1.4 | 1.4 |
| Benchmark | | | | | | | | |

The hospital report also details information collected on the 1,282 patients over a three month period related to: (a) risk factors associated with CAUTIs, and (b) nurse behaviors related to care of Foley catheters. Data were collected for each patient for a period of 1 to 10 days depending on length of stay. Descriptive statistics were calculated in order to evaluate potential risk factors for CAUTI's, including age, gender, weight, stool incontinence, and related nursing practices among patients in critical care.

It should be recognized that sometimes an ICU held few catheterized patients and that multiple variables can impact CAUTI rate, including staff compliance with established protocol. For example, where there are few catheterized patients in the ICU, the CAUTI rate may fall to zero. However, overall, the impact of the protocol of the method of the invention is clearly demonstrated to reduce CAUTI rates and to increase compliance with the new protocol as compared to established protocol.

In order to evaluate nursing practice of Foley care, the following questions were asked of the nurses: 1) was Theraworx® used to clean the perineum? 2) was Theraworx® used to clean the Foley catheter? 3) were the components of the catheter accurately attached? and 4) was the Foley catheter accurately placed? The information relating to nursing practice of Foley care is summarized below in Table 2. The letter “n” refers to the number of device indwelling days.

Example 3: John Muir Medical Center in Walnut Creek, Calif., undertook a study as a quality improvement project in its emergency department, the study undertaken from April 2013 to July 10, 2014, to evaluate the impact of Theraworx® brand antiseptic used in protocols for urinary catheter insertion and maintenance for CAUTI prevention in hospitalized patients. CAUTI's were defined according to the definitions of the Centers for Disease Control and Prevention National Heathcare Safety Network. The John Muir study specifically refers to the Prevention Guidelines of the Healthcare Infection Control Practices Advisory Committee (HICPAC) and to Gould C. V., Umscheid C. A., Agarwal R. K., et al.

The “Guideline for Prevention of Catheter-Associated Urinary Tract Infections 2009,” which was accessed by John Muir Medical Center in 2014, recommends, in

TABLE 2

| | Theraworx | Theraworx | Accurate | Accurate |
|---|---|---|---|---|
| | used to | used to | attachment | placement |
| | cleanse | cleanse | of the | of the |
| Day in | Perineum | Foley | device | Foley |
| Hospital | Yes (n) | Yes (n) | Yes (n) | Yes (n) |
| 1 | 99.1% (1,099) | 99.8% (1,238) | 99.4% (1,238) | 99.8% (1,241) |
| 2 | 98.8% (811) | 100.0% (872) | 99.9% (874) | 99.9% (869) |
| 3 | 99.7% (385) | 99.8% (421) | 99.8% (422) | 100.0% (422) |
| 4 | 97.8% (223) | 99.6% (241) | 99.2% (241) | 100.0% (241) |
| 5 | 100.0% (113) | 100.0% (121) | 100.0% (120) | 100.0% (120) |
| 6 | 100.0% (56) | 100.0% (59) | 100.0% (59) | 100.0% (59) |
| 7 | 100.0% (15) | 100.0% (15) | 100.0% (15) | 100.0% (15) |
| 8 | 100.0% (5) | 100.0% ( [symbol] ) | 100.0% (5) | 100.0% (5) |
| 9 | 100.0% (3) | 100.0% (3) | 100.0% (3) | 100.0% (3) |
| 10 | no data | no data | no data | no data |

[symbol]

indicates data missing or illegible when filed

contrast to the method of the invention studied at John Muir and the subject matter of the invention described herein, that antiseptic solutions not be used for routine catheter maintenance due to a lack of evidence to make an evidence-based decision. However, cleaning the periurethral area with antiseptics is recommended.

The John Muir Medical Center Study concluded that clear trends were evident shortly after use of the full Theraworx protocol was implemented that may show an effective CAUTI prevention intervention once fully implemented that is guideline concordant and fills critical gaps in knowledge.

The John Muir Medical Center protocol included using a cloth impregnated with Theraworx® brand antiseptic to wipe the perineum before Foley catheter insertion, concentrating on the entrance to the meatus, wiping front-to-back for women and in concentric circles around the glans penis for men. This first application was allowed to dry for thirty seconds and not rinsed off. Thereafter, the Foley catheter kit was opened, a Betadine® brand antiseptic swab was used to cleanse the urinary meatus area and the Foley catheter was inserted while practicing accepted sterile techniques. A new, second cloth impregnated with Theraworx® brand antiseptic was used to wipe around the meatus and catheter in a downward direction for post-insertion catheter care, again wiping front-to-back for women and in concentric circles around the glans penis for men. Thereafter, new fresh wipes or a foam solution applied to a clean washcloth were used for routine catheter care and frequent perineum care every 8 to 12 hours, all in accordance with the invention, and for final cleaning after each incidence of incontinence or other contaminating event. In the event high risk factors were identified, then maintenance was increased to every four hours until the catheter was removed.

FIG. 4 graphically illustrates the CAUTI rate per catheter day by month at the Walnut Creek facility for the period of the trial. FIG. 5 graphically illustrates the days between CAUTI's over time, by month.

John Muir Medical Center reported that use of the Theraworx® impregnated cloths in connection with improvement in nursing staff behaviors drastically reduced the number of insertion-related CAUTI's, which are CAUTI's in which a UTI is not present on admission and a positive urine culture develops on or before the third day after insertion. In the four months after the improvements in quality were implemented, the number of documented emergency department related CAUTI went from 3 to less than 1.5 and costs dropped commensurately. In one month, the number of CAUTI's was zero, and no costs attributable to CAUTI were incurred.

Example 4: Euclid Hospital, a hospital in the Cleveland Clinic Hospital system, Cleveland Ohio, undertook a quality improvement project similar to that of John Muir Medical Center from June through August 2014, although June was considered to have been used for training in the protocol of the invention. The CAUTI rate from January 2013 to August 2014 is graphically illustrated in FIG. 6 as the result of a statistical analysis. Euclid Hospital stated that Theraworx® antiseptic solution and the protocol of the invention may be an effective CAUTI prevention intervention and that Theraworx® antiseptic's benefits over other antiseptics includes a broad spectrum of activity, ability to maintain the skin's natural pH, which is to say to protect the stratum corneum, and sufficiently mild for use in the peri-rectal areas and on mucus membranes. Although no special cause variation was identified on the statistical process control chart, FIG. 6, Euclid Hospital was able to reach and maintain zero CAUTI after implementation of the protocol of the invention.

Example 5: First Health Moore Regional Hospital, a 395 bed facility, undertook a quality improvement project for catheter maintenance in all of its ICU's in August 2013 through October 2013 to practice the protocol of the invention. Prior to the study, in the third quarter of 2013, CAUTI rates in the ICU's were about 2.3% per 1,000 catheter days, or 4 CAUTI cases in 1,728 catheter days. These infections were determined to have occurred primarily after a catheter had been in place for more than 5 days and were expected to be due to catheter maintenance, not insertion. The hospital practiced the protocol of the invention, using Theraworx® impregnated cloths, for insertion and maintenance and at the end of October 2013, after 1,667 catheter days, had no CAUTI's and the protocol of the invention was approved for house-wide implementation for catheter insertion and maintenance, despite the current best practice recommended in the most recent literature of soap and water.

Antiseptic solutions for use in the practice of the invention, including Theraworx® brand antiseptic cleanser, unlike cholorohexadrine and alcohol, have no restrictions for application to the face, mucus membranes, the meatus, or perineal and rectal areas, and may be used as frequently as deemed necessary. Formulations of this nature have broad-spectrum antimicrobial activity, anti-yeast and anti-fungal properties, while nourishing and moisturizing the skin, maintaining the natural pH of the skin's mantel, supporting the stratum corneum so that barrier function is preserved even as the skin is decolonized from infectious agents. Prolonged antimicrobial activity is demonstrated up to about three hours. It should be noted that odors are also one indication of infection and that practice of the method of the invention reduces or eliminates odors associated with CAUTI's. The mode of bacterial cellular death is believed to be disruption of cell membranes with the resultant loss of cytoplasmic contents and yet without damage to skin or living tissues. Three substances are believe to contribute: citrus-based antimicrobial stabilizers, zwitterionic surfactants with quaternary ammonium cations, and colloidal silver. The formulation contains vitamin E, aloe vera, allantoin, colloidal silver, and beta glucan 1, and is said to be greater than 99.9% effective against gram negative and gram positive bacteria.

The invention is defined as set forth in the appended claims.

What is claimed is:

1. In a muscle tissue that exhibits a stress response, a method for impacting the stress response of muscle tissue, which muscle tissue is associated with the stratum corneum of intact skin, the stratum corneum having an outermost surface and an innermost surface and defining a thickness therebetween, the method comprising the steps of:
(a) initially applying topically to the outermost surface of the stratum corneum a tissue-compatible antiseptic, fast-acting antimicrobial, non-antibiotic, aqueous composition having a pH of from about 4.0 to about 6.0;
(b) acidifying the stratum corneum throughout its thickness to a substantially uniform pH of from about 4.5 to about 6.0 by continual application of the composition in accordance with step (a) about once every few minutes to once every twelve hours; and
(c) maintaining the acidification of the stratum corneum by repeating step (b) for so long as the muscle tissue exhibits a stress response.

2. The method of claim 1 wherein the composition is radiation-stable and is treated for preservation of activity by exposure to radiation.

3. The method of claim 1 wherein the radiation exposure is from about 4 to 35 kGy gamma radiation.

4. The method of claim 1 wherein the composition is characterized by a sterility assurance level of from about 10·² to 10−⁸.

5. The method of claim 4 wherein the composition is characterized by a sterility assurance level of about 1O−⁶.

6. The method of claim 1 wherein the composition acidifies the skin from the outermost surface of the stratum corneum to the innermost surface of the stratum corneum throughout its thickness at a substantially uniform acidic pH within the range of from about 4.5 to 5.5.

7. The method of claim 1 further comprising the step of warming the composition to increase absorption into the integumentary associated tissues.

8. The method of claim 1 wherein the composition is warmed prior to application to a temperature of from about 100 to 125° F.

9. The method of claim 1 wherein the composition is applied as a foam, spray, liquid, gel or cream, and on a cloth pre-moistened with the composition.

10. The method of claim 1 wherein the composition is allowed to dry after each application without rinsing.

11. The method of claim 1 wherein the composition is treated to preserve activity of active ingredients.

12. The method of claim 1 wherein the composition further comprises glycerin.

13. The method of claim 1 wherein the stress response is a response to exertion and the step (c) of maintaining the acidification of the stratum corneum throughout its thickness by repeating step (b) comprises continual application of the composition in accordance with step (a) every 30 minutes to 2 hours during exertion.

14. The method of claim 1 wherein step (a) is performed at least once prior to exertion.

15. The method of claim 1 wherein application of the composition acidifies and maintains acidification of the stratum corneum throughout its thickness at a substantially uniform pH of from about 4.5 to 5.5.

16. The method of claim 1 wherein the stress response is a response to exertion and the method further comprises further comprising the step step (d) of applying the composition after exertion at least once to maintain acidification of the stratum corneum.

17. The method of claim 1 wherein the stress response is a muscular cramp or tightness and step (c) of maintaining acidification for so long as the muscle tissue exhibits a stress response means until the cramp or tightness is abated.

18. The method of claim 1 wherein the stress response is a muscular cramp or tightness and the intact skin of stratum corneum associated with muscle tissue subject to tightness and cramping is selected from the group consisting of arms, legs, feet and torso.

19. The method of claim 1 wherein the tissue compatible, antiseptic, fast-acting antimicrobial, non-antibiotic, aqueous composition having a pH of from about 4.0 to about 6.0 is comprised of: (a) at least one fast-acting antimicrobial agent; (b) at least one anti-inflammatory agent; and (c) at least one cell growth-promoting agent.

20. The method of claim 1 wherein the tissue compatible, antiseptic, fast-acting antimicrobial, non-antibiotic, aqueous composition having a pH of from about 4.0 to about 6.0 is comprised of: (a) at least one fast-acting antimicrobial agent; (b) at least one anti-inflammatory agent; (c) at least one cell growth-promoting agent; (d) at least one immune system-enhancing agent; (e) at least one free-radical scavenging agent; (g) at least one humectant and emollient agent; and (f) at least one amphoteric surfactant agent.

21. The method of claim 1 wherein the tissue compatible, antiseptic, fast-acting antimicrobial, non-antibiotic, aqueous composition having a pH of from about 4.0 to about 6.0 is comprised of: (a) at least one fast-acting antimicrobial agent; (b) at least one anti-inflammatory agent; (c) at least one cell growth-promoting agent; (d) at least one immune system-enhancing agent; (e) at least one free radical scavenging agent; (f) at least one humectant and emollient agent; and (g) at least one amphoteric surfactant agent (h) at least one anti-foaming agent; (i) at least one absorption-facilitation agent; and G) at least one healing-promoting agent.

22. The method of any of claim 1 wherein the method for impacting the stress response of muscle tissue associated with the stratum corneum of intact skin further comprises impacting the integumentary system associated with the muscle tissue, increasing
oxygenation of muscle tissue, reducing trans-epidermal water loss, speeding clearance of waste by-products of metabolism, and enabling longer and more intensive exercise.

23. The method of claim 1 wherein the tissue compatible, antiseptic, fast-acting antimicrobial, non-antibiotic, aqueous composition having a pH of from about 4.0 to about 6.0 comprises a mixture of ingredients selected from: fast-acting antimicrobial agents, anti-inflammatory agents, cell growth-promoting agents, immune system-enhancing agents, free-radical scavenging agents, humectant and emollient agents, amphoteric surfactant agents, absorption-facilitation agents, and healing-promotion agents.

24. The method of claim 23 wherein the amphoteric surfactant agent is selected from cocamidopropyl betaine, alkyl polyglucosides, lauryl glucosides, and mixtures thereof; the anti-inflammatory agent is selected from aloe vera, allantoin, cocamidopropyl betaine, beta glucan and mixtures thereof; the anti-foaming agent is selected from silicone-based anti-foaming agents, dimethicone copolyol, and mixtures thereof; the cell growth-promoting agent is selected from aloe vera, allantoin, beta glucan, polyphenolic compounds, such as a grapefruit seed extract or bioflavonoid derived quaternary compounds, and mixtures thereof; the fast acting antimicrobial agent is selected from colloidal silver, polyphenolic compounds, such as grapefruit seed extract or bioflavonoid derived quaternary compounds, and mixtures thereof; the immune system-enhancing agent is selected from aloe vera, beta glucan, colloidal silver, allantoin and mixtures thereof; the absorption facilitation agent is selected from beta glucan, aloe vera, colloidal silver and mixtures thereof; the humectant and emollient agent is selected from aloe vera, allantoin, vitamin E, beta glucan, cocamidopropyl betaine and mixtures thereof; the free radical-scavenging agent is selected from polyphenolic compounds, such as grapefruit seed extract or bioflavonoid derived quaternary compounds, beta glucan, allantoin, vitamin E, pycnogenol, grape seed extract and mixtures thereof; and the healing promoting agent is selected from aloe vera, allantoin, polyphenolic compounds, such as grapefruit seed extract or bioflavonoid derived quaternary compounds beta glucan, pharmaceuticals and mixtures thereof.

25. The method of claim 24 wherein the amphoteric surfactant agent is present in an amount of 1 to 7% by weight; the anti-inflamatory agent is present in an amount of 0.2 to 2% by weight; the anti-foaming agent is present in an amount of 0.2 to 2% by weight; the cell growth-promoting agent is present in an amount of 0.1 to 2% by weight; the fast acting antimicrobial agent is present in an amount of 0.4 to 2% by weight; the immune system-enhancing agent is present in an amount of 0.1 to 2% by weight; the cell growth-promoting agent is present in an amount of 0.1 to 2% by weight; the absorption facilitation agent is present in an amount of 0.1 to 4% by weight; the humectant and emollient agent is present in an amount of 0.1 to 6% by weight; the free radical-scavenging agent is present in an amount of 0.1 to 2% by weight; and the healing promoting agent is present in an amount of 0.1 to 2% by weight.

26. The method of claim 19 wherein the fast-acting antimicrobial agent is selected from colloidal silver, polyphenolic compounds, such as grapefruit seed extract or bioflavonoid derived quaternary compounds, and mixtures thereof; the anti-inflammatory agent is selected from aloe vera, allantoin, cocamidopropyl betaine, beta glucan and mixtures thereof; and the cell growth-promoting agent is selected from aloe vera, allantoin, beta glucan, polyphenolic compounds, such as a grapefruit seed extract or bioflavonoid derived quaternary compounds, and mixtures thereof.

27. The method of claim 26 wherein the fast-acting antimicrobial agent is present in an amount of 0.4 to 2% by weight; the anti-inflammatory agent is present in an amount of 0.2 to 2% by weight and the cell growth-promoting agent is present in an amount of 0.1 to 2% by weight.

28. The method of claim 20 wherein the amphoteric surfactant is selected from cocamidopropyl betaine, alkyl polyglucosides, lauryl glucosides, and mixtures thereof; the anti-inflammatory agent is selected from aloe vera, allantoin, cocamidopropyl betaine, and mixtures thereof; the cell growth-promoting agent is selected from aloe vera, allantoin, beta glucan, a bioflavonoid, a polyphenolic compound, a grapefruit derived quaternary compound, and mixtures thereof; the fast acting antimicrobial agent is selected from colloidal silver, a bioflavoniod, a polyphenolic compound, a grapefruit-derived quaternary compound, and mixtures thereof; the one immune system-enhancing agent is selected from aloe vera, beta glucan, colloidal silver, allantoin and mixtures thereof; the humectant and emollient agent is selected from aloe vera, vitamin E, cocamidopropyl betaine, and mixtures thereof; the free radical-scavenging agent is selected from a polyphenolic compound derived from a grapefruit extract quaternary compound or a biflavonoid, beta glucan, allantoin, vitamin E, pycnogenol, grape seed extract, and mixtures thereof.

29. The method of claim 28 wherein the amphoteric surfactant is present in an amount from 1 to 7% by weight; the anti-inflammatory agent is present in an amount from 0.2 to 2% by weight; the cell growth-promoting agent is present in an amount from 0.1 to 2% by weight; the fast acting antimicrobial agent is present in an amount from 0.4 to 2% by weight; the immune system-enhancing agent is present in an amount from 0.1 to 2% by weight; the humectant and emollient agent is present in an amount from 0.1 to 6% by weight; the free radical-scavenging agent is present in an amount from 0.1 to 2% by weight.

30. The method of claim 21 wherein the amophoteric surfactant is selected from cocamidopropyl betaine, alkyl polyglucosides, lauryl glucosides, and mixtures thereof; the anti-inflammatory agent is selected from aloe vera, allantoin, cocamidopropyl betaine, and mixtures thereof; the anti-foaming agent is selected from silicone-based anti-foaming agents, dimethicone copolyol, and mixtures thereof; the cell growth-promoting agent is selected from aloe vera, allantoin, beta glucan, a bioflavonoid, a polyphenolic compound, a grapefruit derived quaternary compound, and mixtures thereof; the fast acting antimicrobial agent is selected from colloidal silver, a bioflavoniod, a polyphenolic compound, a grapefruit-derived quaternary compound, and mixtures thereof; the one immune system-enhancing agent is selected from aloe vera, beta glucan, colloidal silver, allantoin and mixtures thereof; the absorption facilitation agent is selected from beta glucan, aloe vera, colloidal silver, and mixtures thereof; the humectant and emollient agent is selected from aloe vera, vitamin E, cocamidopropyl betaine, and mixtures thereof; the free radical-scavenging agent is selected from a polyphenolic compound derived from a grapefruit extract quaternary compound or a biflavonoid, beta glucan, allantoin, vitamin E, pycnogenol, grape seed extract, and mixtures thereof; and the healing promoting agent is selected from aloe vera, allantoin, beta glucan, and mixtures thereof.

31. The method of claim 30 wherein the amphoteric surfactant is present in an amount from 1 to 7% by weight; the anti-inflammatory agent is present in an amount from 0.2 to 2% by weight; the anti-foaming agent is present in an amount from about 0.2 to 2% by weight; the cell growth-promoting agent is present in an amount from 0.1 to 2% by weight; the fast acting antimicrobial agent is present in an amount from 0.4 to 2% by weight; the immune system
enhancing agent is present in an amount from 0.1 to 2% by weight; the absorption facilitation agent is present in an amount from 0.2 to 4% by weight; the humectant and emollient agent is present in an amount from 0.1 to 6% by weight; the free radical-scavenging agent is present in an amount from 0.1 to 2% by weight; and the healing promoting agent is present in an amount from 0.1 to 2% by weight.
